(19) United States
(12) Patent Application Publication
(10) Pub. No.: US 2022/0000779 A1
(43) Pub. Date: Jan. 6, 2022

(54) IMMUNOGENIC COMPOSITIONS

(71) Applicant: GLAXOSMITHKLINE BIOLOGICALS SA, Rixensart (BE)

(72) Inventors: Geraldine MINET, Rixensart (BE); Dunja SOBOT ERAK, Rixensart (BE); Laurent Bernard Jean STRODIOT, Rixensart (BE)

(73) Assignee: GLAXOSMITHKLINE BIOLOGICALS SA, Rixensart (BE)

(21) Appl. No.: 17/294,214

(22) PCT Filed: Dec. 4, 2019
(86) PCT No.: PCT/EP2019/083747

§ 371 Date: May 14, 2021

(30) Foreign Application Priority Data
Dec. 6, 2018 (EP) 18210871.2

(51) Int. Cl. A61K 9/127; A61K 47/10; A61K 47/24; A61K 39/39; A61K 39/02; A61K 39/08; A61K 39/13; A61K 39/29

(52) U.S. Cl. CPC A61K 9/1272; A61K 47/10; A61K 47/24; A61K 39/39; A61K 39/0208; A61K 2039/55583; A61K 39/13; A61K 39/292; A61K 39/099; A61K 39/02; A61K 39/08

(57) ABSTRACT

Nanoparticles encapsulating polysaccharide conjugates and compositions comprising same are provided. Particularly the nanoparticles are prepared using a microfluidic device.

### FIELD OF THE INVENTION

The present invention relates to lipid nanoparticles comprising polysaccharide conjugates and methods of making same, particularly using microfluidic devices. The invention further relates to compositions comprising the nanoparticles, particularly, immunogenic compositions such as vaccines.

### BACKGROUND TO THE INVENTION

Many medicinal compositions, such vaccines, are combination products which contain two or more biologically-active constituents. It is known that when co-formulated (i.e. formulated together in a single composition such as a parenteral formulation), one constituent may interact with another constituent, such as an adjuvant or an excipient or even water present in the formulation. Such interaction may have a deleterious impact on the biological effect mediated by at least one of the interacting biologically-active constituents (such impact being ‘deleterious’ relative to the biological effect that such biologically-active constituent would mediate if formulated alone, i.e. as the sole biologically-active constituent).

In the case of vaccines, such deleterious interaction may manifest as a physical or biochemical incompatibility, such as an effect on the stability of the biologically-active constituent, and/or as an in vivo phenomenon adversely impacting on the immune response elicited by the constituent (“immunological interference”). For example in the case of paediatric combination vaccines containing Haemophilus influenzae type b (“Hib”) polysaccharide conjugated to a carrier protein (such as tetanus toxoid, “TT”), the Hib antigen may be lyophilised and packaged separately from the liquid, aqueous DTPa/aluminium hydroxide-containing formulation—this is the case in, for example, INFANRIX Hexa (GSK Vaccines).

There are two reasons for this: first, because the Hib-derived polysaccharide part (polyribosylribitol, “PRP”) of the Hib conjugate antigen is labile to degradation when in aqueous formulation, particularly in the presence of Al(OH)₃; and secondly because the PRP can interact with aluminium hydroxide to form a network of particles (“flocculation”) which may mask PRP epitopes from the recipient's immune system. In the case of vaccines such as INFANRIX Hexa, partitioning the vaccine components between a liquid, aqueous component and a lyophilised component, which are extemporaneously reconstituted at the time of administration, is an acceptable solution to overcome the above problems. However, it leads to a two-part vaccine requiring a reconstitution step to be carried out by the medical personnel administering the vaccine. WO2017/153316 discloses methods of encapsulating biological agents to overcome this issue. EP1928418 discloses encapsulation of polysaccharide antigens and protein carriers that are specifically not conjugated to one another.

However, there is a need to develop new formulations and further methods that can overcome problems associated with interaction of constituents within a composition. Particularly, these methods should be industrially scaleable and efficient.

### SUMMARY OF THE INVENTION

The present invention provides nanoparticles comprising a lipid component and a polysaccharide conjugate component. The nanoparticles are particularly useful for encapsulating and protecting polysaccharide conjugates from interaction with the external environment.

Thus, in a first aspect of the invention there is provided a nanoparticle comprising a lipid component and a polysaccharide conjugate component. Particularly the nanoparticle of the invention is a liposome. Liposomes are sealed vesicles having a lipid membrane. Liposomes may have different membrane structures, for example, unilamellar liposomes are vesicles that comprise a single lipid membrane whilst multilamellar liposomes comprise several lipid membranes. Particularly, the lipid membrane is a lipid bilayer. Liposomes generally exist in the form of a suspension in which an internal aqueous phase is contained or encapsulated within at least one lipid bilayer thereby being isolated or separated from the external environment, for example, an external aqueous phase. Nanoparticles of the invention may be unilamellar or multilamellar. A population of nanoparticles may comprise both unilamellar and multilamellar liposomes.

Particularly the lipid component comprises 1,2-Dioleoyl-sn-Glycero-3-Phosphocholine (DOPC; CAS Number: 4235-95-4). More particularly the lipid component comprises Cholesterol (CAS Number: 57-88-5). Yet more particularly the lipid component comprises Polyethylene glycol (PEG; CAS Number: 25322-68-3). Yet more particularly a phospholipid-PEG conjugate, for example, 1, 2-Distearoyl-sn-glycero-3-phosphoethanolamine-Poly(ethylene glycol) (DSPE-PEG; CAS Number: 474922-26-4). Still yet more particularly the lipid component comprises a cationic lipid. In certain embodiments the cationic lipid is 1,2-dioleoyltrimethylammonium propane (DOTAP; CAS Number: 144189-73-1). In certain embodiments the cationic lipid is dimethyldioctadecylammonium bromide (DDAB; CAS Number: 3700-67-2). In some embodiments of the invention, the lipid component comprises (i) DOPC, (ii) at least one cationic lipid, (iii) Cholesterol and (iv) PEG or DSPE-PEG. In some embodiments of the invention, the lipid component comprises (i) DOPC, (ii) at least one cationic lipid selected from the group consisting of DOTAP and DDAB, (iii) Cholesterol and (iv) PEG or DSPE-PEG. Thus in more particular embodiments of the invention, the lipid component comprises (i) DOPC, (ii) DOTAP, (iii) Cholesterol and (iv) PEG or DSPE-PEG. In other more particular embodiments of the invention, the lipid component comprises (i) DOPC, (ii) DDAB, (iii) Cholesterol and (iv) PEG or DSPE-PEG. In certain embodiments the lipid component comprises OPC:DOTAP:Cholesterol:DSPE-PEG in a ratio (molar concentration %, mol %) of about 4:60:34:2. In other embodiments the lipid component comprises DOPC:DDAB:Cholesterol:DSPE-PEG in a ratio (molar concentration %, mol %) of about 4:60:34:2.

The polysaccharide conjugate component comprises (i) a polysaccharide and (ii) a carrier. Preferably the carrier is covalently bound to the polysaccharide. In certain embodiments the carrier is covalently bound to the polysaccharide via a linker, for example, adipic acid dihydrazide. Particularly the polysaccharide is a bacterial capsular polysaccharide. The polysaccharide may be naturally occurring or a synthetic polysaccharide, more particularly a synthetic polysaccharide that mimics elements of the structure of the naturally occurring capsular polysaccharide. Particularly the polysaccharide is polyribosylribitol phosphate (PRP), for example, from Haemophilus influenzae serotype B (Hib). Particularly the polyribosylribitol phosphate is a synthetic polysaccharide capable of cross-reacting with antibodies specific to naturally occurring polyribosylribitol phosphate derived from Haemophilus influenzae serotype B, for example as disclosed in WO2018/020046. The carrier may be a protein, for example, tetanus toxoid (TT), diphtheria toxoid (DT), CRM197, OMPC or an outer membrane vesicle such as a dOMV, nOMV or GMMA.

In a second aspect of the invention there is provided a plurality of nanoparticles according to the first aspect. Particularly the average nanoparticle size, i.e. diameter, is from about 150 nm to about 250 nm. More particularly, the average nanoparticle size, i.e. diameter, is determined by dynamic light scattering or electron microscopy. Particularly the plurality of nanoparticles has a polydispersity index in the range of from about 0.2 to 0.45. More particularly, the polydispersity index is determined by dynamic light scattering.

In a third aspect of the invention, there is provided an aqueous composition comprising the population of nanoparticles according to the second aspect. The polysaccharide conjugate may be present at a concentration of about 50 μg/ml or less, for example, the polysaccharide conjugate may be present at a concentration of about 30 μg/ml, 31 μg/ml, 32 μg/ml, 33 μg/ml, 34 μg/ml, 35 μg/ml, 36 μg/m1 37 μg/ml, 38 μg/ml, 39 μg/ml or about 40 μg/ml. In some embodiments, the polysaccharide conjugate may be present at a concentration of more than 50 μg/ml The lipid component may be present at a concentration of about 10 mg/ml. Particularly the lipid:polysaccharide ratio (w/w) is about 200:1. In some embodiments the aqueous composition is an immunogenic composition, particularly a vaccine. In some embodiments, the aqueous composition comprises at least one antigen selected from the group consisting of diphtheria toxoid (DT), tetanus toxoid (TT), inactivated polio virus (IPV), hepatitis B surface antigen, pertussis toxoid (PT), pertactin, filamentous hemagglutinin (FHA) and fimbrial protein.

In a fourth aspect of the invention, there is provided a nanoparticle of the first aspect, the plurality of nanoparticles of the second aspect or the aqueous composition of the third aspect, for use in a method of raising an immune response in a mammal. Particularly the mammal is a human.

In a fifth aspect, there is provided a method of manufacturing a nanoparticle according to the first aspect, a population of nanoparticles according to the second aspect or an aqueous composition according to the third aspect, using a microfluidic device, the method comprising the steps of mixing in the device a first solution comprising an aqueous solvent and a polysaccharide conjugate and a second solution comprising an organic solvent and (i) DOPC, (ii) DOTAP or DDAB, (iii) Cholesterol and (iv) PEG or DSPE-PEG. Particularly the method may be used to manufacture nanoparticles of the invention on a commercially viable scale.

### DESCRIPTION OF FIGURES

FIG. 1 shows the design of the microfluidic device utilized in the examples.

FIG. 2 provides data relating to size measurements of the nanoparticles (PS=polysaccharide concentration; PL=lipid concentration).

FIG. 3 provides details of the set up of the flocculation experiment.

FIG. 4: demonstrates results obtained in the presence of alum—no flocculation is observed for Hib-TT encapsulated with the DOTAP or DDAB containing compositions.

FIG. 5: Demonstrates that, with regard to flocculation, the compositions are stable over time.

FIG. 6: Both candidates showed spherical liposome-like structure by cryo-EM (A=DOPC-Cholesterol-DOTAP-PEG/50 μg/mL Hib, average hydrodynamic diameter ˜155 nm; B=DOPC-Cholesterol-DDAB-PEG/50 μg/mL Hib, average hydrodynamic diameter ˜211 nm).

FIG. 7: Tabular summary of the in vivo DDAB liposome studies.

FIG. 8: Immunogenicity study of Hib-TT encapsulated in DDAB based liposomes: overview of serology results at 7PII—Anti-Hib (anti-PRP) IgG serology data.

FIG. 9: (a) A significant difference between group 1 and 3 is indicative of interference and validates the experiment model; (b) at 7PII, Hib-TT immunogenicity was reduced in the presence of DDAB; (c) Similar levels of response were observed following immunisation with Hib-TT encapsulated in DDAB either mixed or co-administered with Infanrix Penta. The level of response was also similar to that obtained following extemporaneous mixing of Hib-TT with Infanrix Penta; (d) No differences were observed between compositions mixed extemporaneously or injected after 4 weeks.

FIG. 10: Immunogenicity study of Hib-TT encapsulated in DDAB based liposomes: overview of serology results at 7PII—Anti-Hib (anti-PRP) IgG serology data.

FIG. 11: (a) A significant difference between group 1 and 3 was indicative of interference and also validated the experiment model at 7PIII; (b) at 7PIII, the level of anti-Hib response was similar between Hib-TT encapsulated in DDAB or admixed with empty DDAB and Hiberix given in co-administration with Infanrix Penta; (c) Similar levels of response were observed following immunisation with Hib-TT encapsulated in DDAB either mixed or co-administered with Infanrix Penta. The level of response was also similar to that obtained following extemporaneous mixing of Hib-TT with Infanrix Penta; (d) No differences were observed between compositions mixed extemporaneously or injected after 4 weeks.

FIG. 12: Immunogenicity of Hib-TT encapsulated in DDAB based liposomes: comparison of serology results obtained at 7PII and 7PIII. The level of response at 7PIII was increased when compared to 7PII in all groups that received DDAB. FIG. 13: SBA titers (pooled serum) at 7PIII against Haemophilus influenzae vaccine strain 20.752.

FIG. 14: Tabular summary of the in vivo DOTAP liposome studies.

FIG. 15: Immunogenicity study of Hib-TT encapsulated in DOTAP based liposomes: overview of serology results at 7PII—Anti-Hib (anti-PRP) IgG serology data.

FIG. 16: (a) A significant difference between group 1 and 3 is indicative of interference and validates the experiment model; (b) a similar level of response was observed between Hib-TT non ads and Hib-TT liquid bulk used for encapsulation indicating that any differences are not due to the Hib-TT used for encapsulation; (c) at 7P11, Hib-TT immunogenicity was reduced in the presence of DOTAP; (d) Similar levels of response were seen in rats that received Infanrix Penta/empty DOTAP liposomes co-administered with Hib-TT liquid bulk and groups immunized in co-administration of either with Hib-TT lyo or liquid bulk (without DOTAP); (e) No significant differences were observed between groups immunised with Hib-TT mixed with empty DOTAP liposomes or Hib-TT encapsulated within DOTAP liposomes; (f) similar level of responses were observed when Hib-TT encapsulated in DOTAP liposomes either mixed with or following co-administration with Infanrix Penta. The level of response was similar to that of Group 1.

FIG. 17: Immunogenicity study of Hib-TT encapsulated in DOTAP based liposomes: overview of serology results at 7PIII—Anti-Hib (anti-PRP) IgG serology data.

FIG. 18: (a) A significant difference between group 1 and 3 is indicative of interference and confirmed the experiment model at 7PIII; (b) a similar level of response was observed between Hib-TT non ads and Hib-TT liquid bulk used for encapsulation indicating that any differences are not due to the Hib-TT used for encapsulation; (c) In contrast to what was observed at the 7PII timepoint, at 7PIII Hib-TT immunogenicity was no longer reduced in the presence of DOTAP liposomes when co-administered. The level of anti-Hib response was similar between Hib-TT encapsulated in DOTAP liposomes or admixed with empty DOTAP liposomes and Hib TT co-administered with Infanrix Penta; (d) as was the case at 7P11, at 7PIII the addition of empty DOTAP liposomes to Infanrix Penta did not impact the anti-Hib response; (e) the level of response observed following immunisation with Hib-TT encapsulated in DOTAP liposomes either mixed or co-administered with Infanrix Penta was similar; (f) lower responses were observed in the group immunised with Hib-TT and empty DOTAP liposomes compared to the group immunised with Hib-TT encapsulated in DOTAP liposomes indicative of an added value of encapsulation.

FIG. 19: Immunogenicity of Hib-TT encapsulated in DOTAP based liposomes: comparison of serology results obtained at 7PII and 7PIII. The level of response at 7PIII was increased when compared to 7PII in all groups that received DOTAP.

FIG. 20: SBA titers (pooled serum) at 7PIII against Haemophilus influenzae vaccine strain 20.752.

### DETAILED DESCRIPTION OF THE INVENTION

By “lipid” is intended a class of organic compounds that are fatty acids or their derivatives and are insoluble in water but soluble in organic solvents, including natural oils, waxes, and steroids (for example, sterols, including cholesterol).

By “liposome” is intended a microvesicle composed of one or more bilayers of lipidic amphipathic molecules that may enclose one or more aqueous compartments.

The term “polysaccharide” is intended to include naturally occurring polysaccharides as well as polysaccharides that are obtained via chemical synthesis or genetic engineering. The term is used to include disaccharides, oligosaccharides and longer saccharide polymers, wherein the individual monomeric saccharide units may be naturally occurring or modified.

Some polysaccharide conjugates may be prone to hydrolytic degradation over time or sensitive to catalytic reactions, for example, between phosphodiester bonds and some aluminum adjuvants. Encapsulation has been found to protect polysaccharide conjugates from degradation. However, the encapsulating matrices used in the art may be bioincompatible and the methods used may be complex or ineffificient at larger scales.

The inventors have developed new matrices for encapsulation of polysaccharide conjugates. Thus, the present invention relates to nanoparticles within which at least one polysaccharide conjugate is encapsulated thereby protecting the conjugate from interaction with the external environment. Advantageously, the nanoparticles may be prepared using microfluidic devices

Nanoparticles of the invention may be referred to as liposomes. As discussed above, liposomes may be unilammelar or multilammelar. Multilammelar liposomes have multiple bilayers within each vesicle forming several separate aqueous compartments. In contrast, unilammelar liposomes have a single bilayer encapsulating an aqueous core. Liposomes of the invention ideally have a diameter in the range of about from 50 nm to about 300 nm, for example about 100 nm to about 250 nm, such as, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm or 250 nm. For compositions comprising a plurality of liposomes with different diameters the average diameter of at least 70%, at least 75%, at least 80%, at least 85%, at least 90% or at least 95% should be in the range of about from 50 nm to about 220 nm, for example about 100 nm to about 220 nm, such as, 100 nm, 110 nm, 120 nm, 130 nm, 140 nm, 150 nm, 160 nm, 170 nm, 180 nm, 190 nm, 200 nm or 220 nm. If the liposomes are not spherical, the term “diameter” refers to the largest cross-sectional diameter of the nanoparticle.

For compositions comprising a plurality of liposomes, the plurality of liposomes should have a polydispersity index of from about 0.01 to about 0.45, for example, 0.05, 0.1, 0.15, 0.2, 0.25, 0.3, 0.35 or 0.4. The term “polydispersity” (or “dispersity” as recommended by IUPAC) is used to describe the degree of non-uniformity of a size distribution of a population of particles. The “Polydispersity Index” (PDI) is a dimensionless parameter used in the art to to define the size distribution of the lipid nanoparticles. The higher the value of the polydispersity index, the broader the spread of individual particle sizes making up the population. The lower the value of the polydispersity index, the more uniform and tightly grouped the particle sizes of the individual nanoparticles making up the population will be. The PDI may be analysed using dynamic light scattering (DLS) techniques known in the art.

Nanoparticles of the invention are formed from a mixture of lipids, for example, at least two or at least three different lipids. Particularly the lipid component comprises a helper lipid, a sterol and a cationic lipid. A suitable helper lipid is dioleoylphosphatidylcholine (DOPC), a suitable sterol is cholesterol and suitable cationic lipids include 1,2-dioleoyltrimethylammonium propane (DOTAP) and dimethyldioctadecylammonium bromide (DDAB). The lipid component may also comprise Polyethylene glycol (PEG) or a PEG conjugate, for example, a poly(ethylene glycol phospholipid conjugate such as DSPE-PEG. Particular combinations of lipid components comprise: (i) DOPC, (ii) DOTAP, (iii) Cholesterol and (iv) DSPE-PEG or (i) DOPC, (ii) DDAB, (iii) Cholesterol and (iv) DSPE-PEG. With regard to the lipid component, the molar ratio (molar concentration %) is the ratio of the mass of one constituent of the lipid component to the mass of the other constituents of the lipid component. In certain embodiments the lipid component comprises DOPC:DOTAP:Cholesterol:DSPE-PEG in a molar ratio (molar concentration %) of A:B:C:D. In other embodiments the lipid component comprises DOPC:DDAB:Cholesterol:DSPE-PEG in a ratio (w/w) of A:B:C:D. A, B, C or D are integers selected from about 1 to 65, for example, 2, 3, 4, 5, 6, 7, 8, 9, 10, 15, 20, 25, 30, 31, 32, 33, 34, 35, 36, 37, 38, 39, 40, 45, 50, 55, 56, 57, 58, 59, 60, 61, 62, 63, 64 or 65. A preferred molar ratio (molar concentration %) of A:B:C:D is about 4:60:34:2.

The polysaccharide conjugate component comprises (i) a polysaccharide and (ii) a carrier. The term “polysaccharide conjugate” refers to a polysaccharide linked covalently to a carrier, such as a protein. In some embodiments the polysaccharide is directly linked to a carrier. In other embodiments the polysaccharide is indirectly linked to a carrier through a spacer or linker. As used herein, the term “directly linked” means that the two entities are connected via a chemical bond, preferably a covalent bond. As used herein, the term “indirectly linked” means that the two entities are connected via a linking moiety (as opposed to a direct covalent bond). In certain embodiments the linker is adipic acid dihydrazide.

Covalent linkage of polysaccharides to proteins is known in the art and is generally achieved by targeting the amines of lysines, the carboxylic groups of aspartic/glutamic acids or the sulfhydryls of cysteines. For example, cyanate esters randomly formed from sugar hydroxyls can be reacted with the lysines of the protein or the hydrazine of a spacer which are then condensed to the carboxylic acids of the carrier protein via carbodiimide chemistry. Alternatively, aldehydes generated on purified polysaccharide by random periodate oxidation can either be used directly for reductive amination onto the amines of the carrier, such as a protein, or converted into amines for following insertion of a spacer enabling the conjugation step to the protein via thioether or amide bond formation. Glycoconjugates obtained by these methods present complex cross-linked structures. A strategy aimed at simplifying the structure of the final conjugate employs partial hydrolysis of the purified polysaccharide and following fractionation to select an intermediate chain length population. A primary amino group can then be introduced at the oligosaccharide reducing termini to be used finally for insertion of either a diester or a bifunctional linker ready for conjugation to the carrier. Preferably the carrier is covalently bound to the polysaccharide. In certain embodiments the carrier is covalently bound to the polysaccharide via a linker, for example, adipic acid dihydrazide.

The terms “protein carrier” or “carrier protein” are used interchangeably and refer to a protein to which the polysaccharide is coupled or attached or conjugated, typically for the purpose of enhancing or facilitating detection of the antigen by the immune system. Polysaccharides, such as bacterial capsular polysaccharides, are generally T-independent antigens that are poorly immunogenic and do not induce long-term protective immune responses. Conjugation of a polysaccharide antigen to a protein carrier changes the context in which immune effector cells respond to polysaccharides. The term carrier protein is intended to cover both small peptides and large polypeptides (>10 kDa). The carrier protein may comprise one or more T-helper epitopes. The peptide may be coupled to the carrier protein by any means such as chemical conjugation.

Useful carrier proteins are known in the art and may include bacterial toxins or toxoids, such as diphtheria toxoid or tetanus toxoid. The CRM 197 mutant of diphtheria toxin may also be used. Other suitable carrier proteins include the N. meningitidis outer membrane protein complex (OMPC), human serum albumin (preferably recombinant), protein D from H. influenzae, pneumococcal surface protein PspA, pneumolysin, and the like. Particularly suitable carrier proteins include CRM 197, tetanus toxoid (TT), tetanus toxoid fragment C, protein D, non-toxic mutants of tetanus toxin and diphtheria toxoid (DT). In some embodiments, the carrier is an outer membrane vesicle, for example, a dOMV, nOMV or GM MA.

Particularly the polysaccharide is a bacterial capsular polysaccharide. The polysaccharide may be naturally occurring or a synthetic polysaccharide, more particularly a synthetic polysaccharide that mimics elements of the structure of the naturally occurring capsular polysaccharide. In other words, the polysaccharide is produced by synthetic, chemical processes but is capable of mimicking the native polysaccharide in antigenic and immunogenic properties such that it is capable of inducing an immune response against a particular bacteria. Particularly the polysaccharide is polyribosylribitol phosphate (PRP), for example, from Haemophilus influenzae serotype B (Hib). Particularly the polyribosylribitol phosphate is a synthetic polysaccharide capable of cross-reacting with antibodies specific to naturally occurring polyribosylribitol phosphate derived from Haemophilus influenzae serotype B. In other embodiments, the polysaccharide is the capsular polysaccharide of Group A Neisseria meningitidis or a synthetic mimic thereof. Methods for preparing polysaccharides and polysaccharide conjugates are known in the art. Representative polysaccharide conjugates for use in accordance with the present invention include Hib PRP-TT, Hib PRP-DT, Hib PRP-CRM197, Hib PRP-OMPC, MenA-TT, MenA-DT, MenA-CRM197 and MenA-OMPC.

The liposomes may comprise some external polysaccharide conjugate (e.g. on their surface), but preferably at least 50% of the polysaccharide conjugate, for example, at least 60%, at least 70%, at least 80%, at least 85%, at least 90%, at least 91%, at least 92%, at least 93%, at least 94%, at least 95%, at least 96%, at least 97%, at least 98%, at least 99% is encapsulated within the core of the nanoparticle or liposome.

### Immunogenic Compositions

Nanoparticles, such as liposomes, of the invention are useful as components in immunogenic compositions for immunising subjects against various diseases. Thus, in a third aspect of the invention, there is provided an aqueous composition comprising at least one nanoparticle of the first aspect or the population of nanoparticles according to the second aspect.

The term “immunogenic composition” broadly refers to any composition that may be administered to elicit an immune response, such as an antibody or cellular immune response, against an antigen present in the composition. Thus compositions of the invention are immunogenic. Immunogenic compositions may be referred to as pharmaceutical compositions. When the immunogenic compositions prevent, ameliorate, palliate or eliminate disease from the subject, then such compositions may be referred to as a vaccine. Vaccines according to the invention may either be prophylactic (i.e. to prevent infection) or therapeutic (i.e. to treat infection), but will typically be prophylactic. The term “to prevent infection” means that a subject's immune system has been sensitized (for example, by vaccination) to elicit an immune response and repel the infection. It will be obvious to those skilled in the art that a vaccinated subject may thus become infected but is more likely to repel the infection, for example more quickly, than a control subject. In certain embodiments, the immunogenic composition is a vaccine. The term “antigen” refers to a substance that, when administered to a subject, elicits an immune response directed against the substance. In the context of the present invention, the polysaccharide and polysaccharide conjugate are antigens. Preferably the polysaccharide is a synthetic polysaccharide prepared or manufactured using synthetic chemistry techniques. Particularly, when administered to a subject the immunogenic composition elicits an immune response directed against the polysaccharide. Particularly the immune response directed against the polysaccharide is protective, that is, it can prevent or reduce infection or colonisation caused by a bacteria, for example, Haemophilus influenzae or Neisseria meningitidis.

The polysaccharide conjugate may be present at a concentration of about 50 μg/ml. For example, the polysaccharide is present at at a concentration of from about 1 μg/ml to about 30 μg/ml, for example from 2 μg/ml to 25 μg/ml, and in particular from about 5 μg/ml to about 20 μg/ml, particular values include about 10 μg/ml, about 5 μg/ml or about 1 μg/ml for example, about 10 μg/dose, about 5 μg/dose, about 2.5 pg/dose, about 1 μg/dose or about 0.5 μg/dose. Pharmaceutical compositions of the invention may be prepared in unit dose form. In some embodiments a unit dose may have a volume of from 0.1 ml to about 1.0 ml, for example, about 0.5 ml. Suitable amounts of polysaccharide may include about 1 μg, about 2.5 μg, about 5 μg, about 10 μg and about 20 μg per unit dose. Each dose may be about 0.5 ml. In certain embodiments, each 0.5 mL dose contains about 10 μg of Haemophilus influenzae type b polysaccharide or synthetic polysaccharide conjugated to about 25 μg of carrier, for example a carrier protein such as tetanus toxoid. Compositions of the invention are preferably sterile. Immunogenic compositions of the invention are preferably non-pyrogenic e.g. containing <1 ED (endotoxin unit, a standard measure) per dose, and preferably <0.1 EU per dose. Pharmaceutical compositions of the invention are preferably gluten free.

The lipid component may be present at a concentration of about 1 mg/ml to about 50 mg/ml, for example, 5 mg/ml, 10 mg/ml, 15 mg/ml, 20 mg/ml, 25 mg/ml or 30 mg/ml. Particularly the lipid component is present at a concentration of about 10 mg/ml Particularly compositions of the invention comprise an excess of lipid:polysaccharide. For example, the lipid:polysaccharide ratio (w/w) is from about 10:1 to about 250:1, for example about 20:1, 30:1, 40:1, 50:1, 60:1, 70:1, 80:1, 90:1, 100:1, 125:1, 150:1, 175:1 or about 200:1. Particularly, the lipid:polysaccharide ratio (w/w) is from about 200:1

In some embodiments, the aqueous composition comprises at least one additional antigen (i.e. in addition to the polysaccharide conjugate), particularly, selected from the group consisting of diphtheria toxoid (DT), tetanus toxoid (TT), inactivated polio virus (IPV), hepatitis B surface antigen, pertussis toxoid (PT), pertactin, FHA and fimbrial protein. Reference to diptheria toxoid and tetanus toxoid in the context of additional antigens is intended to mean that the additional antigen(s) is present is free, unconjugated form distinct from any carrier that might be present in the conjugate.

Immunogenic compositions of the invention will generally comprise a pharmaceutically acceptable excipient in addition to the nanoparticles. A “pharmaceutically acceptable excipient” is an excipient that does not itself induce the production of antibodies. Such pharmaceutically acceptable excipients are well known to those of ordinary skill in the art and include, by way of non-limiting example, polysaccharides, polylactic acids, polyglycolic acids, amino acid copolymers, sucrose, trehalose, lactose and the like. Immunogenic compositions may also contain diluents, such as water, saline or glycerol (which may also be isotonicity agents), and the like. Sterile pyrogen-free, phosphate-buffered physiologic saline is a typical diluent. Such compositions may also include, auxiliary substances, such as wetting or emulsifying agents, pH buffering substances, etc. The pH of the composition may be between pH 6 and pH 8, particularly about pH 7. Stable pH may be maintained by the use of a buffer. Compositions of the invention may include the liposomes in plain water (e.g. w.f.i.) or in a buffer such as a phosphate buffer, a Tris buffer, a borate buffer, a succinate buffer, a histidine buffer, or a citrate buffer. Buffer salts will typically be included in the 5-20 mM range. Compositions of the invention may have a pH between 5.0 and 9.5 e.g. between 6.0 and 0. Compositions of the invention may include sodium salts (e.g. sodium chloride) to give tonicity. A concentration of 10±2 mg/ml NaCl is typical e.g. about 9 mg/m L. Compositions of the invention may have an osmolality of between 200 mOsm/kg and 750 mOsm/kg, e.g. between 240-360 mOsm/kg, or between 290-310 mOsm/kg. Compositions of the invention may be hypotonic or mildly hypertonic. Compositions of the invention may include one or more preservatives, such as thiomersal or 2-phenoxyethanol. Mercury-free compositions are preferred, and preservative-free vaccines can be prepared.

Suitable immunogenic compositions may be in aqueous form, for example, as a solution or suspension or in a dried form, for example, lyophilised. Dried or lyophilised compositions are generally reconstituted with a liquid medium prior to injection. For lyophilisation, a stabiliser such as a sugar alcohol (e.g. mannitol) and/or a disaccharide (e.g. sucrose or trehalose) may be included. Immunogenic compositions are preferably sterile and may also be pyrogen-free. Compositions may be isotonic with respect to the subject's body. Preferably the immunogenic composition is an aqueous composition.

Immunogenic compositions may be prepared in various forms, in vials or as injectables in ready filled syringes, either with or without needles. Syringes generally contain a single dose of the composition, whilst a vial may contain a single dose or multiple doses. Compositions may be prepared for pulmonary administration, for example, as a fine powder or a spray for administration using an inhaler. Other forms for administration are known to the skilled person including, by way of non-limiting example, solid dosage forms, suppositories and pessaries, compositions for nasal, aural or ocular administration such as sprays, drops, gels or powders.

Immunogenic compositions used as vaccines comprise an immunologically effective amount of antigens. The term “immunologically effective amount” refers to the amount of an antigen or antigens needed to stimulate or achieve the desired immunologic effect, particularly a cellular (T cell) response, a humoral (B cell or antibody) response, or both, as measured by standard assays known to one skilled in the art. This amount may vary depending upon the health and physical condition of the subject to be treated, age, capacity of the individual's immune system to synthesise antibodies, degree of protection desired, formulation and the like. One skilled in the art understands that the immunologically effective amount is the amount of antigen administered to a subject in a single dose and that the amount can be determined through routine trials, such as clinical or dose-ranging trials, and may fall within a range.

The polysaccharide content of compositions of the invention will generally be expressed in terms of the amount of polysaccharide per dose. The amount of polysaccharide in a single dose of immunogenic composition will generally be in the range of from 1 μg (0.001 mg) to 120 μg (0.120 mg). More particularly in the range of from about 2.5 μg (0.0025 mg) to about 50 μg (0.05 mg), for example, in the range of from about 5 μg (0.005 mg) to about 50 μg (0.05 mg), for example about 9.5, 9.6, 9.7, 9.8, 9.9, 10,10.1, 10.2, 10.3, 10.4, 10.5, 11, 11.5, 12, 12.5, 13, 13.5, 14, 14.5, 15, 20, 25, 30, 35, 40, 45 or 50 μg of polysaccharide. The skilled person understands that in a multi-component immunogenic composition, i.e. one containing at least two different antigens, the immunologically effective amount of each antigen is likely to be different and therefore represents a proportion of the total amount of protein antigen per dose. By way of non-limiting example, an immunogenic composition that comprises an immunologically effective amount, Xpg, of a first antigen and an immunologically effective amount, Ypg, of a second antigen will comprise X+Y_μg of total antigen per dose.

Immunogenic compositions will generally comprise one or more adjuvants. As used herein, “adjuvant” means a compound or substance (or combination of compounds or substances) that, when administered to a subject in conjunction with an antigen or antigens, for example as part of an immunogenic composition or vaccine, increases or enhances the subject's immune response to the administered antigen or antigens (compared to the immune response obtained in the absence of adjuvant).

Suitable adjuvants include an aluminum salt such as aluminum hydroxide gel or aluminum phosphate or alum, but may also be a salt of calcium, magnesium, iron or zinc, or may be an insoluble suspension of acylated tyrosine, or acylated sugars, cationically or anionically derivatized saccharides, or polyphosphazenes. In some embodiments, the adjuvant is selected from the group consisting of aluminium phosphate, aluminium hydroxide and alum.

Suitable adjuvant systems which promote a predominantly Th1 response include: non-toxic derivatives of lipid A, Monophosphoryl lipid A (MPL) or a derivative thereof, particularly 3-de-O-acylated monophosphoryl lipid A (3D-MPL) (for its preparation see GB 2220211 A); and a combination of monophosphoryl lipid A, preferably 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen (Thoelen et al. Vaccine (1998) 16:708-14; EP 689454-B1).

AS01 is an Adjuvant System containing MPL (3-O-desacyl-4′-monophosphoryl lipid A), QS21 ((Quillaja saponaria Molina, fraction 21) Antigenics, New York, N.Y., USA) and liposomes. AS01B is an Adjuvant System containing MPL, QS21 and liposomes (50 μg MPL and 50 μg QS21). ASO1E is an Adjuvant System containing MPL, QS21 and liposomes (25 μg MPL and 25 μg QS21). In one embodiment, the immunogenic composition or vaccine comprises AS01. In another embodiment, the immunogenic composition or vaccine comprises AS01B or ASO1E. In a particular embodiment, the immunogenic composition or vaccine comprises ASO1E. AS02 is an Adjuvant Aystem containing MPL and QS21 in an oil/water emulsion. ASO2V is an Adjuvant System containing MPL and QS21 in an oil/water emulsion (50 μg MPL and 50 μg QS21). AS03 is an Adjuvant System containing α-Tocopherol and squalene in an oil/water (o/w) emulsion. ASO3A is an Adjuvant System containing α-Tocopherol and squalene in an o/w emulsion (11.86 mg tocopherol). AS03B is an Adjuvant System containing a-Tocopherol and squalene in an o/w emulsion (5.93 mg tocopherol). AS03C is an Adjuvant System containing a-Tocopherol and squalene in an o/w emulsion (2.97 mg tocopherol). In one embodiment, the immunogenic composition or vaccine comprises AS03. AS04 is an Adjuvant System containing MPL (50 μg MPL) adsorbed on an aluminum salt (500 μg Al3+). In one embodiment, the immunogenic composition or vaccine comprises AS04. A system involving the use of QS21 and 3D-MPL is disclosed in WO 94/00153. A composition wherein the QS21 is quenched with cholesterol is disclosed in WO 96/33739. An additional adjuvant formulation involving QS21, 3D-MPL and tocopherol in an oil in water emulsion is described in WO 95/17210. In one embodiment the immunogenic composition additionally comprises a saponin, which may be QS21. The formulation may also comprise an oil in water emulsion and tocopherol (WO 95/17210). Unmethylated CpG containing oligonucleotides (WO 96/02555) and other immunomodulatory oligonucleotides (WO 0226757 and WO 03507822) are also preferential inducers of a TH1 response and are suitable for use in the present invention.

Additional adjuvants are those selected from the group of metal salts, oil in water emulsions, Toll like receptor agonists, (in particular Toll like receptor 2 agonist, Toll like receptor 3 agonist, Toll like receptor 4 agonist, Toll like receptor 7 agonist, Toll like receptor 8 agonist and Toll like receptor 9 agonist), saponins or combinations thereof. Compositions of the invention may include one or more small molecule immunopotentiators. For example, the composition may include a TLR2 agonist (e.g. Pam3CSK4), a TLR4 agonist (e.g. an aminoalkyl glucosaminide phosphate, such as E6020), a TLR7 agonist (e.g. imiquimod), a TLR8 agonist (e.g. resiquimod (also a TLR7 agonist)) and/or a TLR9 agonist (e.g. IC31). Any such agonist ideally has a molecular weight of <2000 Da. In some embodiments such agonist(s) are also encapsulated with the polysaccharide conjugate in liposomes, but in other embodiments they are unencapsulated.

Possible excipients include arginine, pluronic acid and/or polysorbate. In a preferred embodiment, polysorbate 80 (for example, TWEEN (a US registered trademark) 80) is used. In a further embodiment, a final concentration of about 0.03% to about 0.06% is used. Specifically, a final concentration of about 0.03%, 0.04%, 0.05% or 0.06% polysorbate 80 (w/v) may be used.

Formulations comprising the immunogenic compositions of the invention may be adapted for administration by an appropriate route, for example, by the intramuscular, sublingual, transcutaneous, intradermal or intranasal route. Such formulations may be prepared by any method known in the art.

The invention also provides a device (e.g. a vial) or delivery device (e.g. syringe, nebuliser, sprayer, inhaler, dermal patch, etc.) containing an immunogenic composition of the invention. This device can be used to administer the composition to a vertebrate subject.

### Methods of Treatment and Medical Uses

Nanoparticles of the invention, particularly plurality of nanoparticles according to the invention including aqueous compositions thereof may be used for eliciting an immune response against an immunogen of interest, particularly the polysaccharide. As disclosed herein, methods for raising an immune response in a vertebrate comprising the step of administering an effective amount of a nanoparticle or aqueous composition of the invention are provided. The immune response is preferably protective and preferably involves antibodies and/or cell mediated immunity. The method may raise a booster response.

The invention also provides a nanoparticle or aqueous composition of the invention for use in a method for raising an immune response in a vertebrate.

The invention also provides the use of a nanoparticle or aqueous composition of the invention in the manufacture of a medicament for raising an immune response in a vertebrate.

By raising an immune response in the vertebrate by these uses and methods, the vertebrate can be protected against various diseases and/or infections e.g. against bacterial diseases as discussed above. The liposomes and compositions are immunogenic, and are more preferably vaccine compositions. Vaccines according to the invention may either be prophylactic (i.e. to prevent infection) or therapeutic (i.e. to treat infection), but will typically be prophylactic.

The vertebrate is preferably a mammal, particularly a suitable mammal, such as a human or a large veterinary mammal (e.g. horses, cattle, deer, goats, and pigs). Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

Vaccines prepared according to the invention may be used to treat both children and adults. Thus a human patient may be less than 1 year old, less than 5 years old, 1-5 years old, 5-15 years old, 15-55 years old, or at least 55 years old. Preferred patients for receiving the vaccines are the elderly (e.g. 20 ˜50 years old, ˜60 years old, and preferably ˜65 years), the young (e.g. ˜5 years old), hospitalised patients, healthcare workers, armed service and military personnel, pregnant women, the chronically ill, or immunodeficient patients. The vaccines are not suitable solely for these groups, however, and may be used more generally in a population.

Compositions of the invention will generally be administered directly to a patient. Direct delivery may be accomplished by parenteral injection (e.g. subcutaneously, intraperitoneally, intravenously, intramuscularly, intradermally, or to the interstitial space of a tissue. Alternative delivery routes include rectal, oral (e.g. tablet, spray), buccal, sublingual, vaginal, topical, transdermal or transcutaneous, intranasal, ocular, aural, pulmonary or other mucosal administration. Intradermal and intramuscular administration are two preferred routes. Injection may be via a needle (e.g. a hypodermic needle), but needle-free injection may alternatively be used. A typical intramuscular dose is 0.5 mL.

The invention may be used to elicit systemic and/or mucosal immunity, preferably to elicit an enhanced systemic and/or mucosal immunity.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.). In one embodiment, multiple doses may be administered approximately 6 weeks, 10 weeks and 14 weeks after birth, e.g. at an age of 6 weeks, 10 weeks and 14 weeks, as often used in the World Health Organisation's Expanded Program on Immunisation (“EPI”). In an alternative embodiment, two primary doses are administered about two months apart, e.g. about 7, 8 or 9 weeks apart, followed by one or more booster doses about 6 months to 1 year after the second primary dose, e.g. about 6, 8, 10 or 12 months after the second primary dose. In a further embodiment, three primary doses are administered about two months apart, e.g. about 7, 8 or 9 weeks apart, followed by one or more booster doses about 6 months to 1 year after the third primary dose, e.g. about 6, 8, 10, or 12 months after the third primary dose.

### Methods of Manufacture

The invention also provides a method of manufacturing a nanoparticle according to the first aspect, a population of nanoparticles according to the second aspect, or an aqueous composition according to the third aspect. Preferably the method uses a microfluidic device and comprises the steps of mixing in the device a first solution comprising an aqueous solvent and a polysaccharide conjugate and a second solution comprising an organic solvent and (i) DOPC, (ii) DOTAP or DDAB, (iii) Cholesterol and (iv) PEG or DSPE-PEG.

A microfluidic device is a fluid handing apparatus having at least one dimension on a sub-millimetre scale and typically mixing occurs through passive means (i.e. through contact of fluid streams and without moving parts within the mixing chamber). The microfluidic device will comprise a mixing chamber within which the first solution and second solution are mixed.

The mixing chamber will typically have a cross-sectional area which is 25.6 mm² or less, such as 12.8 mm² or less, suitably 6.4 mm² or less, especially 3.2 mm² or less and in particular 1.6 mm² or less. The mixing chamber will typically have a cross-sectional area which is 0.1 mm² or more, suitably 0.2 mm² or more, especially 0.3 mm² or more and in particular 0.4 mm² or more. In some embodiments the mixing chamber will have a cross-sectional area which is 0.2-3.2 mm², such as 0.4-1.6 mm², especially 0.6-1.2 mm² and in particular 0.7-1.0 mm² (e.g. 0.8 mm²).

The cross-section of the mixing chamber may be of any shape, though is typically symmetrical. The cross-section may be substantially rectangular (such as square). The cross-section may be elongate in nature, with the larger dimension being at least twice that of the perpendicular dimension, such as at least three times or at least four times. The larger dimension may be no more than ten times that of the perpendicular dimension, such as no more than eight times or no more than six times. The larger dimension will usually be two to ten times that of the perpendicular dimension, such as three to eight times, especially four to six times, in particular five times.

A rectangular cross-section may have a long side of 1-8 mm, such as 1-4 mm, for example 1.4-3.2 mm, especially 1.6-2.4 mm, in particular 1.8-2.2 mm (e.g. 2 mm). A rectangular cross-section may have a short side of 0.1 to 4 mm, for example, 0.1 to 2 mm, optionally 0.1-1.2 mm, such as 0.1-0.8 mm, especially 0.2-0.6 mm, in particular 0.3-0.5 mm (e.g. 0.4 mm).

The microfluidic device will have at least one inlet (such as one inlet) to the mixing chamber for delivery of the first solution. The device may have a plurality of inlets to the mixing chamber for delivery of the first solution, such as two inlets. The microfluidic device will have at least one inlet to the mixing chamber for delivery of the second solution. The device may have a plurality of inlets to the mixing chamber for delivery of the second solution, such as two inlets. To facilitate adequate mixing, the number of inlets for the first solution and second solution may be increased for mixing chambers with larger cross-sectional areas.

The cross-section of the inlets may be of any shape, though is typically symmetrical. The cross-section may be rectangular (such as square).

Each inlet will typically have a cross-sectional area which is 1.28 mm² or less, suitably 0.64 mm² or less, especially 0.32 mm² or less and in particular 0.16 mm² or less. Each inlet will typically have a cross-sectional area which is 0.01 mm² or more, suitably 0.02 mm² or more, especially 0.03 mm² or more and in particular 0.04 mm² or more. In some embodiments each inlet will have a cross-sectional area which is 0.02-0.32 mm², such as 0.04-0.16 mm², especially 0.06-0.12 mm² and in particular 0.07-0.10 mm² (e.g. 0.8 mm²). The total cross-sectional area of all inlets will suitably be less than 70% of the cross-sectional area of the mixing chamber, such as less than 60% and especially less than 50%. Conveniently, the inlets may span the full length of one side of the mixing chamber.

The shape and size of each inlet may be varied independently. However, typically inlets for the first solution will be identical in shape and size, and inlets for the second solution will be identical in shape and size. Conveniently, all inlets are identical in shape and size. A particular inlet design is rectangular in shape, 0.2 mm wide and spanning the full length of the other side of the mixing chamber (e.g. 0.4 mm high)

The inlets will typically be located such that the direction of flow of the first solution and second solution into the mixing chamber is substantially parallel (e.g. within 15 degrees, such as within 10 degrees, in particular within 5 degrees), such as parallel, to the general direction of flow through the mixing chamber.

The microfluidic device will have at least one outlet from the mixing chamber for recovery of the mixed material. The device may have a plurality of outlets from the mixing chamber for recovery of the mixed material, such as two or three outlets, which are later combined. Suitably the device will have a single outlet from the mixing chamber for recovery of the mixed material.

The cross-section of the outlets may be of any shape, though is typically symmetrical. The cross-section may be rectangular (such as square), typically having an area of 0.2-1 mm2, such as 0.3-0.6 mm2, for example 0.4-0.5 mm2. In other examples the outlet may be of circular cross-section (e.g. having a diameter of 0.5-1 mm, such as 0.6-0.8 mm, for example 0.75 mm).

The total cross-sectional area of all outlets will suitably be less than 70% of the cross-sectional area of the mixing chamber, such as less than 60% and especially less than 50%.

The mixing chamber should be of adequate length to allow for mixing to be substantially complete by the time liquid reaches the outlet(s). Typically, the chamber will be 1-10 cm in length, such as 1.5-5 cm, especially 1.8-4 cm, in particular 2-3 cm, for example 2.5 cm.

In one embodiment the device comprises a mixing chamber which is rectangular in cross-section, having a cross-sectional area of 0.2-3.2 mm2 (e.g. 0.6-1.0 mm2), a long side of 1.4-3.2 mm (e.g. 1.6-2.4 mm), a short side of 0.1-1.2 mm (e.g. 0.32-0.48 mm), one inlet for the first solution and two inlets for the second solution which are symmetrically disposed at the proximal end of the mixing chamber, a mixing chamber length of 1.5-5 cm (e.g. 2-3 cm) and an outlet located at the distal end of the mixing chamber. Suitably the inlets are 0.16-0.24 mm wide and span the full length of the other side of the mixing chamber.

The microfluidic device may be formed from any suitable material, namely one which is tolerant of the components used in the first solution and second solution and which is amenable to manufacture. Suitable materials include silicon and glass. Devices may be prepared from such materials by etching, e.g. silicon devices may be prepared by Deep Reactive Ion Etching (DRIE or plasma etching) and glass devices may be prepared by wet etching (HF etching).

To achieve a batch run duration which is a manageable time period (e.g. 240 minutes or less, especially 120 minutes or less) it is necessary for the system to achieve a sufficient level of productivity. Additionally, to aid batch to batch consistency by reducing the impact of start up and shutdown effects it is necessary for the run time to be of adequate length (e.g. at least 30 minutes, especially at least 60 minutes).

### Methods for Encapsulating Polysaccharides by Microfluidics

The lipids utilized in the methods herein may be prepared by solubilizing individual lipids in solvent and combining the appropriate amount to produce a stock solution of total lipids comprising the calculated percent, ratio, or weight of each lipid. Alternatively, the lipids utilized in the methods herein may be prepared by combining the appropriate amount of each lipid and then solubilising them in solvent. Particularly the second solution comprising an organic solvent and (i) DOPC, (ii) DOTAP or DDAB, (iii) Cholesterol and (iv) PEG or DSPE-PEG is prepared by combining the appropriate amount of each lipid, for example as a powder and then solubilising them in solvent. Codisolution of lipid powders may be advantageous for improving the solubility of certain lipids.

The stock solution of lipids plus solvent for use herein is prepared at a convenient concentration of lipids. Advantageously, by increasing the stock solution concentration one may work at a lower volume before nanoprecipitation and the final product can be more concentrated. In some embodiments, the solution comprising solvent further comprises at least 1 mg/mL, at least 2 mg/mL, at least 3 mg/mL, at least 4 mg/mL, at least 5 mg/mL, at least 6 mg/mL, at least 7 mg/mL, at least 8 mg/mL, at least 9 mg/mL, at least 10 mg/mL, at least 15 mg/mL, at least 20 mg/mL of total lipid. In some embodiments, wherein the solution comprising solvent further comprises between from 1-20 mg/mL, from 1-15 mg/mL, from1-10 mg/mL of total lipid, but no more than 50 mg/mL of total lipid.

The solvent utilized in the solution of lipids is compatible with lipids and miscible with the aqueous solution. In some embodiments, the solvent in the solution of lipids may be a Class 3 solvent, including acetic acid, heptane, acetone, isobutyl acetate, anisole, isopropyl acetate, 1-butanol, methyl acetate, 2-butanol, 3-methyl-1-butanol, butyl acetate, methylethyl ketone, tert-butylmethyl ether, 2-methyl-1-propanol, dimethyl sulfoxide, pentane, ethanol, 1-pentanol, ethyl acetate, 1-propanol, ethyl ether, 2-propanol, ethyl formate, propyl acetate, formic acid, and triethylamine. In some embodiments, the solvent in the solution of lipids may be an organic alcohol. In some embodiments, the solvent comprises between 70-100% ethanol. In some embodiments, the solvent is at least 80%, at least 90%, at least 95%, at least 98%, at least 99% organic alcohol. In some embodiments, the solvent is less than 10%, less than 5%, less than 4%, less than 3%, less than 2%, less than 1%, less than 0.5% water. In some embodiments, the solvent in the solution of lipids is selected from the group consisting of isopropanol and ethanol. In some embodiments, the solvent comprises between 70-100% ethanol. In some embodiments, the solvent is at least 80%, at least 90%, at least 95%, at least 98%, at least 99% ethanol. In some embodiments, the ethanol comprises less than 10%, less than 5%, less than 4%, less than 3%, less than 2%, less than 1%, less than 0.5% water. In some embodiments, the solvent is 100% ethanol.

The methods of the invention involve (1) mixing a first solution comprising an aqueous solvent and a polysaccharide conjugate and a second solution comprising an organic solvent and (i) DOPC, (ii) DOTAP or DDAB, (iii) Cholesterol and (iv) PEG or DSPE-PEG, and (2) removing the solvent. In some embodiments the microfluidic device comprises 2-128 mixing chambers. Particularly the aqueous solvent comprises a buffer, particularly a maleate buffer. The maleate buffer may have a concentration of from about 10 mM to about 100 mM. The Maleate buffer may have a pH of about 6.

The first and second solutions used in the methods herein may be mixed at proportions that successfully produce nanoparticles, particularly liposomes, having acceptable physico-chemical characteristics. Further, the methods herein may be utilized at specific temperatures and/or flow rates to enhance the physico-chemical characteristics of the nanoparticles produced.

The ratio of aqueous to organic components used herein may be adjusted to successfully produce nanoparticles having acceptable physico-chemical characteristics. In some embodiments, the ratio of water (i.e., aqueous solution) to organic solvent is between 1:1 and 5:1 v/v; between 1.25:1 and 4:1 v/v; between 1.5 and 3:1 v/v. In some embodiments, the ratio of water to organic solvent is about 1.4:1 v/v; about 2:1 v/v; or about 3:1 v/v. In some embodiments, the ratio of water to organic solvent is about 2:1 v/v. In some embodiments, the organic solvent is ethanol, and the ratio of water to ethanol is between from 1:1 and 5:1 v/v, between from 1.25:1 and 4:1 v/v, between from 1.5 and 3:1 v/v. In some embodiments, the ratio of water to ethanol is about 1.4:1; about 2:1; or about 3:1. In some embodiments, the ratio of water to ethanol is about 2:1.

By controlling the total flow rate (TFR) in the microfluidic device, one may successfully produce nanoparticles having acceptable physico-chemical characteristics. In some embodiments, a TFR in the device of greater than 8 ml/min/mm² successfully produces nanoparticles having acceptable physico-chemical characteristics. In some embodiments, the TFR is between 8-30 mL/min/mm², 12-28 mL/min/mm², 14-26 mL/min/mm², 16-24 mL/min/mm², or about 18 mL/min/mm² or about 22 mL/min/mm². Particularly, the TFR is about 16 mL/min/mm². Particularly the flow rate of the aqueous phase (mL/min) is about 12.8. Yet more particularly the flow rate of the organic phase (comprising lipids) (mL/min) is about 3.2.

The temperature of the solution or solutions within the device may also be adjusted to successfully produce nanoparticles having acceptable physico-chemical characteristics. In some embodiments, the temperature of the solution in the microfluidic device is between 10° C. and 37° C., between 15° C. and 36° C., between 20° C. and 35° C., between 25° C. and 34° C., between 30° C. and 33° C., or about 30° C.

In some embodiments, the use of the methods above produces a nanoparticle, such as a liposome, with an average size of from about 150 nm to about 250 nm. In some embodiments, the use of the methods above produces a liposome with a polydispersity in the range of from about 0.2 to about 0.45, for example, of 0.45 or less, 0.4 or less, 0.3 or less, 0.2 or less, or about 0.2.

### Additional Process Steps

In some embodiments, the solvent is removed by buffer exchange, diafiltration, ultrafiltration, dialysis, or a combination thereof. In some embodiments, solvent removal results in a water content of at least 95%; at least 96%; at least 97%; at least 98%; at least 99% at least 99.5% water v/v. In some embodiments, the methods described above are followed by an additional step of diluting, such as to a desired final concentration. In some embodiments, the methods described above are followed by the additional step of sterilization by filtration.

Thus, there is provided a method of manufacturing a nanoparticle according to the first aspect, a population of nanoparticles according to the second aspect, or an aqueous composition according to the third aspect, using a microfluidic device, the method comprising the steps of: (1) mixing in the device a first solution comprising an aqueous solvent and a polysaccharide conjugate and a second solution comprising an organic solvent and (i) DOPC, (ii) DOTAP or DDAB, (iii) Cholesterol and (iv) PEG or DSPE-PEG, and (2) removing the solvent. Particularly the solvent is ethanol. Particularly the solvent is removed by buffer exchange, diafiltration, ultrafiltration, dialysis, or a combination thereof. More particularly solvent removal results in a water content of at least 98% water w/w. The method may comprise the additional step of (3) diluting to a desired concentration. The method may comprise the additional step of (4) sterilisation by filtration. The method may comprise the additional step of (5) formulating the nanoparticles into an immunogenic composition.

Specific Embodiments of the Invention

Embodiment 1: A method of manufacturing a nanoparticle, such as a liposome, encapsulating a polysaccharide conjugate using a microfluidic device, comprising the steps of (i) mixing in the device (a) a first solution comprising an aqueous solvent and the polysaccharide conjugate; and (b) a second solution comprising DOPC, a sterol, a cationic lipid, and a PEGylated lipid; and (ii) removing the solvent.

Embodiment 2: The method of embodiment 1, wherein the solvent comprises an organic alcohol.

Embodiment 3: The method of any preceding Embodiment, wherein the solvent comprises 70-100% ethanol.

Embodiment 4: The method of any preceding Embodiment, wherein 30-60% (mole percent) of total lipids in the solution comprising solvent are cationic.

Embodiment 5: The method of any preceding Embodiment, wherein about 35%, about 40%, about 45%, about 50%, about 55%, about 60% (mole percent) of total lipids in the solution comprising solvent are cationic, for example, DOTAP or DDAB.

Embodiment 6: The method of any preceding Embodiment, wherein 20-40% (mole percent) of total lipids in the solution comprising solvent are cholesterol.

Embodiment 7:The method of any preceding Embodiment, wherein 0.5-5% (mole percent) of total lipids in the solution comprising solvent are a PEGylated lipid for example DSPE-PEG.

Embodiment 8: The method of any preceding Embodiment, wherein 1.0-3.0% (mole percent) of total lipids in the solution comprising solvent are a 1, 2-Distearoyl-sn-glycero-3-phosphoethanolamine-Poly(ethylene glycol) (DSPE-PEG).

Embodiment 9: The method of any preceding Embodiment, wherein about 1.5%, about 1.6%, about 1.7%, about 1.8%, about 1.9%, about 2.0%, about 2.5% (mole percent) of total lipids in the solution comprising solvent are 1,2-Distearoyl-sn-glycero-3-phosphoethanolamine-Poly(ethylene glycol) (DSPE-PEG)

Embodiment 10: The method of any preceding Embodiment, wherein 2-15% (mole percent) of total lipids in the solution comprising solvent are DOPC.

Embodiment 11: The method of any preceding Embodiment, wherein 4-10% (mole percent) of total lipids in the solution comprising solvent are DOPC.

Embodiment 12: The method of any preceding Embodiment, wherein about 4%, about 5%, about 6%, about 7%, about 8% (mole percent) of total lipids in the solution comprising solvent are DOPC.

Embodiment 13: The method of any preceding Embodiment, wherein the solution comprising solvent further comprises at least 1 mg/mL; at least 2 mg/mL; at least 3 mg/mL; at least 4 mg/mL; at least 5 mg/mL; at least 6 mg/mL; at least 7 mg/mL; at least 8 mg/mL; at least 9 mg/mL; at least 10 mg/mL; at least 15 mg/mL; at least 20 mg/mL of total lipid.

Embodiment 14: The method of any preceding Embodiment, wherein the solution comprising solvent further comprises between 1-20 mg/mL; 1-15 mg/mL; 1-10 mg/mL of total lipid.

Embodiment 15: The method of any preceding Embodiment, wherein the microfluidic device comprises a plurality of mixing chambers, such as 2-128 mixing chambers or 4-32 mixing chambers.

Embodiment 16: The method of any preceding Embodiment, wherein the device comprises 16 mixing chambers.

Embodiment 17: The method of any one of Embodiments 14-16, wherein all mixing chambers in the plurality of mixing chambers are supplied by the same pumps and mixed material from all mixing chambers is collected before further processing and/or storage.

Embodiment 18: The method of any one of Embodiments 14-17, wherein the plurality of mixing chambers is capable of producing mixed material at a rate of 50-2000 ml/min.

Embodiment 19: The method of any preceding Embodiment, wherein the average liposome size is 140 nm or lower, 130 nm or lower, 120 nm or lower, or 100 nm or lower.

Embodiment 20: The method of any preceding Embodiment, wherein the liposome polydispersity is 0.4 or less, 0.3 or less, or about 0.2.

Embodiment 21: The method of any preceding Embodiment, wherein the solvent is removed by buffer exchange, diafiltration, ultrafiltration, dialysis, or a combination thereof.

Embodiment 22: The method of any preceding Embodiment, wherein solvent removal results in a water content of at least 98% water w/w.

Embodiment 23: The method of any preceding Embodiment, comprising the additional step of diluting, such as to a desired final concentration.

Embodiment 24: The method of any preceding Embodiment, comprising the additional step of sterilization by filtration.

Embodiment 25: The method of any preceding Embodiment, wherein the total flow rate is 8-30 mL/min/mm², for example, about 12-28 mL/min/mm², particularly about 16 mL/min/mm².

Embodiment 26: A nanoparticle comprising a lipid component and a polysaccharide conjugate component, said lipid component comprising (i) DOPC, (ii) a cationic lipi, (iii) Cholesterol and (iv) a PEGylated lipid.

Embodiment 27: The nanoparticle of Embodiment 26, wherein 30-60% (mole percent) of total lipids in the lipid component are cationic.

Embodiment 28: The nanoparticle of Embodiment 26 or 27, wherein about 35%, about 40%, about 45%, about 50%, about 55%, about 60% (mole percent) of total lipids in the lipid component are cationic, for example, DOTAP or DDAB.

Embodiment 29: The nanoparticle of Embodiment 26-28, wherein 20-40% (mole percent) of total lipids in the lipid component are cholesterol.

Embodiment 30: The nanoparticle of Embodiment 26-29, wherein 0.5-5% (mole percent) of total lipids in the lipid component are a PEGylated lipid for example DSPE-PEG.

Embodiment 31: The nanoparticle of Embodiment 26-30, wherein 1.0-3.0% (mole percent) of total lipids in the lipid component are a 1, 2-Distearoyl-sn-glycero-3-phosphoethanolamine-Poly(ethylene glycol) (DSPE-PEG).

Embodiment 32: The nanoparticle of Embodiment 26-31, wherein about 1.5%, about 1.6%, about 1.7%, about 1.8%, about 1.9%, about 2.0%, about 2.5% (mole percent) of total lipids in the lipid component are 1,2-Distearoyl-sn-glycero-3-phosphoethanolamine-Poly(ethylene glycol) (DSPE-PEG)

Embodiment 33: The nanoparticle of Embodiment 26-32, wherein 2-15% (mole percent) of total lipids in the lipid component are DOPC.

Embodiment 34: The nanoparticle of Embodiment 26-33, wherein 4-10% (mole percent) of total lipids in the lipid component are DOPC.

Embodiment 35: The nanoparticle of Embodiment 26-34, wherein about 4%, about 5%, about 6%, about 7%, about 8% (mole percent) of total lipids in the lipid component are DOPC.

### General

The term “comprising” encompasses “including” e.g. a composition “comprising” X may include something additional e.g. X+Y. The word “substantially” does not exclude “completely” e.g. a composition which is “substantially free” from Y may be completely free from Y. In some implementations, the term “comprising” refers to the inclusion of the indicated active agent, such as recited polypeptides, as well as inclusion of other active agents, and pharmaceutically acceptable carriers, excipients, emollients, stabilizers, etc., as are known in the pharmaceutical industry. In some implementations, the term “consisting essentially of” refers to a composition, whose only active ingredient is the indicated active ingredient(s), for example antigens, however, other compounds may be included which are for stabilizing, preserving, etc. the formulation, but are not involved directly in the therapeutic effect of the indicated active ingredient. Use of the transitional phrase “consisting essentially” means that the scope of a claim is to be interpreted to encompass the specified materials or steps recited in the claim, and those that do not materially affect the basic and novel characteristic(s) of the claimed invention. See, In re Herz, 537 F.2d 549, 551-52, 190 USPQ 461, 463 (COPA 1976) (emphasis in the original); see also MPEP § 2111.03. Thus, the term “consisting essentially of” when used in a claim of this invention is not intended to be interpreted to be equivalent to “comprising”. The term “consisting of” and variations thereof means limited to” unless expressly specified otherwise. In certain territories, the term “comprising an active ingredient consisting of” may be used in place of “consisting essentially”. The term “about” in relation to a numerical value x means, for example, x±10%, x±5%, x±4%, x±3%, x±2%, x±1%, The word “substantially” does not exclude “completely” e.g. a composition which is “substantially free” from Y may be completely free from Y. Where necessary, the word “substantially” may be omitted from the definition of the invention. Where ranges are referred to as ‘between X and Y’, such ranges are generally intended to also encompass the specific values of X and Y as end-points. Where methods refer to steps of administration, for example as (a), (b), (c), etc., these are intended to be sequential, i.e., step (c) follows step (b) which is preceded by step (a). Antibodies will generally be specific for their target, i.e., they will have a higher affinity for the target than for an irrelevant control protein, such as bovine serum albumin.

Embodiments herein relating to “vaccine compositions” of the invention are also applicable to embodiments relating to “immunogenic compositions” of the invention, and vice versa.

All references or patent applications cited within this patent specification are incorporated by reference herein.

### EXAMPLES

### Example 1

The principles of flash-nanoprecipitation were applied to the problem of developing manufacturing approaches which enable the safe, convenient and cost effective production of liposome-encapsulated polysaccharide conjugates on a commercially viable scale while preserving the physicochemical characteristics that maintain immunological performance arising from conventional manufacturing approaches. Flash-nanoprecipitation involves two miscible fluids that are mixed inside a microchip to induce a precipitation. Lipids are solubilized in an organic phase (solvent) and mixed with polysaccharide conjugates, such as the Hib PRP conjugate, in water based phase (anti solvent) in order to make particles.

TABLE 1
Materials.
Materials

| Material and Instrument | Vendor |
|---|---|
| Ethanol absolute alcohol, without | Merck |
| additive, ≥99.8% | |
| Cholesterol (low endotoxin) | Sigma-Aldrich |
| Water for Injection (WFI) | LASO |
| DOPC (PC 18:1/18:1), under Argon | LIPOID |
| DOTAP (18:1 TAP) Chloride salt | Avanti Polar Lipids |
| (under Argon) | |
| DDAB (18:0 TAP) Bromide salt | Avanti Polar Lipids |
| PEG2 PE (Ammonium salt) 18:0 | Avanti Polar Lipids |
| Mealeate (Na) 100 mM, pH 6.0 | LASO |
| Maleate (Na) 10 mM, pH 6.0 | LASO |
| Hib TT conjugate | GSK Vaccines |
| Slide-A-Lyser Dialysis Cassette | Thermo Scientific |

### Equipment

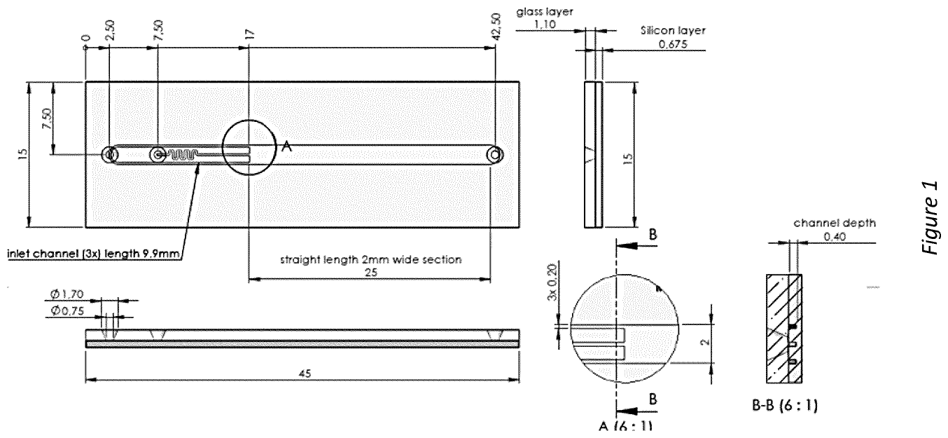

The schematic for a microchip (from Micronit Microtechnologies™) used in the examples herein is shown in FIG. 1. Two mid pressure Nemesys™ pumps were connected to the microchip via a chip-holder available from MicroNit. Tubing connections were diameter: IDEX 1528L 1/16×0.030 ft. One was used for the organic phase containing the lipid mixture (connected to center channel of the microfluidic). The second was used for the aqueous phase containing maleate buffer and the polysaccharide conjugate (connected to the external channels). The Nemesys pumps were controlled by the “neMESYS Userinterface™” installed on a computer.

Seven different compositions were prepared and tested, compositions 1-4 were prepared at Room temperature whilst compositions 5-7 were prepared at 60° C. Composition 1, was a control with no HibTT added:

| | | | final | final conc in | conc in final | | final V |
|---|---|---|---|---|---|---|---|
| | mol | MW | conc | org phase | formulation | mass | org ph |
| lipid | (%) | (g/mol) | (mM) | (mg/mL) | (mg/mL) | (%) | (mL) |
| DOPC | 4.30 | 786.113 | 0.7181 | 2.822538727 | 0.564507745 | 6 | |
| Cholesterol | 33.70 | 386.7 | 5.6279 | 10.88154465 | 2.17630893 | 22 | |
| DDAB | 60 | 630.952 | 10.02 | 31.6106952 | 6.32213904 | 63 | |
| DSPE-PEG | 2 | 2805.497 | 0.334 | 4.68517999 | 0.937035998 | 9 | |
| total (all | 100.00 | 598.801899 | 16.7 | 49.99995857 | 9.999991713 | 100 | 3 |
| lipids) | | | | | | | |

| | | | final | final conc in | conc in final | | final V |
|---|---|---|---|---|---|---|---|
| | mol | MW | conc | org phase | formulation | mass | org ph |
| lipid | (%) | (g/mol) | (mM) | (mg/mL) | (mg/mL) | (%) | (mL) |
| DOPC | 64.00 | 786.113 | 9.2672 | 36.42533197 | 7.285066394 | 73 | |
| Cholesterol | 34.00 | 386.7 | 4.9232 | 9.5190072 | 1.90380144 | 19 | |
| DSPE-PEG | 2 | 2805.497 | 0.2896 | 4.062359656 | 0.812471931 | 8 | |
| total (all | 100.00 | 690.70026 | 14.48 | 50.00669882 | 10.00133976 | 100 | 3 |
| lipids) | | | | | | | |

| | | | final | final conc in | conc in final | | final V |
|---|---|---|---|---|---|---|---|
| | mol | MW | conc | org phase | formulation | mass | org ph |
| lipid | (%) | (g/mol) | (mM) | (mg/mL) | (mg/mL) | (%) | (mL) |
| DOPC | 4.30 | 786.113 | 0.67252 | 2.643383574 | 0.528676715 | 5 | |
| Cholesterol | 33.70 | 386.7 | 5.27068 | 10.19085978 | 2.038171956 | 20 | |
| DOTAP | 60 | 698.5 | 9.384 | 32.77362 | 6.554724 | 66 | |
| DSPE-PEG | 2 | 2805.497 | 0.3128 | 4.387797308 | 0.877559462 | 9 | |
| total (all | 100.00 | 639.330699 | 15.64 | 49.99566066 | 9.999132132 | 100 | 3 |
| lipids) | | | | | | | |

| | | | final | final conc in | conc in final | | final V |
|---|---|---|---|---|---|---|---|
| | mol | MW | conc | org phase | formulation | mass | org ph |
| lipid | (%) | (g/mol) | (mM) | (mg/mL) | (mg/mL) | (%) | (mL) |
| DOPC | 4.30 | 786.113 | 0.7181 | 2.822538727 | 0.564507745 | 6 | |
| Cholesterol | 33.70 | 386.7 | 5.6279 | 10.88154465 | 2.17630893 | 22 | |
| DDAB | 60 | 630.952 | 10.02 | 31.6106952 | 6.32213904 | 63 | |
| DSPE-PEG | 2 | 2805.497 | 0.334 | 4.68517999 | 0.937035998 | 9 | |
| total (all | 100.00 | 598.801899 | 16.7 | 49.99995857 | 9.999991713 | 100 | 3 |
| lipids) | | | | | | | |

| | | | final | final conc in | conc in final | | final V |
|---|---|---|---|---|---|---|---|
| | mol | MW | conc | org phase | formulation | mass | org ph |
| lipid | (%) | (g/mol) | (mM) | (mg/mL) | (mg/mL) | (%) | (mL) |
| DOPC | 4.30 | 786.113 | 0.7181 | 2.822538727 | 0.564507745 | 6 | |
| Cholesterol | 33.70 | 386.7 | 5.6279 | 10.88154465 | 2.17630893 | 22 | |
| DDAB | 60 | 630.952 | 10.02 | 31.6106952 | 6.32213904 | 63 | |
| DSPE-PEG | 2 | 2805.497 | 0.334 | 4.68517999 | 0.937035998 | 9 | |
| total (all | 100.00 | 598.801899 | 16.7 | 49.99995857 | 9.999991713 | 100 | 3 |
| lipids) | | | | | | | |

| | | | final | final conc in | conc in final | | final V |
|---|---|---|---|---|---|---|---|
| | mol | MW | conc | org phase | formulation | mass | org ph |
| lipid | (%) | (g/mol) | (mM) | (mg/mL) | (mg/mL) | (%) | (mL) |
| DOPC | 4.30 | 786.113 | 0.7181 | 2.822538727 | 0.564507745 | 6 | |
| Cholesterol | 33.70 | 386.7 | 5.6279 | 10.88154465 | 2.17630893 | 22 | |
| DDAB | 60 | 630.952 | 10.02 | 31.6106952 | 6.32213904 | 63 | |
| DSPE-PEG | 2 | 2805.497 | 0.334 | 4.68517999 | 0.937035998 | 9 | |
| total (all | 100.00 | 598.801899 | 16.7 | 49.99995857 | 9.999991713 | 100 | 3 |
| lipids) | | | | | | | |

| | | | final | final conc in | conc in final | | final V |
|---|---|---|---|---|---|---|---|
| | mol | MW | conc | org phase | formulation | mass | org ph |
| lipid | (%) | (g/mol) | (mM) | (mg/mL) | (mg/mL) | (%) | (mL) |
| DOPC | 4.30 | 786.113 | 0.67252 | 2.643383574 | 0.528676715 | 5 | |
| Cholesterol | 33.70 | 386.7 | 5.27068 | 10.19085978 | 2.038171956 | 20 | |
| DOTAP | 60 | 698.5 | 9.384 | 32.77362 | 6.554724 | 66 | |
| DSPE-PEG | 2 | 2805.497 | 0.3128 | 4.387797308 | 0.877559462 | 9 | |
| total (all | 100.00 | 639.330699 | 15.64 | 49.99566066 | 9.999132132 | 100 | 3 |
| lipids) | | | | | | | |

Lipid solutions, described above, in organic phase were prepared by weighing each of the lipid powders in the same vial and solubilising with ethanol to obtain a solution. The use of codissolution of the lipid powders was found to be advantageous increasing the solubility of certain lipids. Aqueous solution of Hib-TT was prepared in 10 mM maleate (pH6).

### Microfluidics

A cleaning step was done before using the microfluidics system. Ethanol was loaded to both ports (polysaccharide and Lipid port) by filling up a syringe with ethanol and delivering ethanol to the microchip to rinse the system completely. This was repeated twice. Then the polysaccharide port was loaded with maleate buffer and lipid port with ethanol by filling a syringe with the relevant solution and then delivering either the aqueous or organic solution to the microchip. This was repeated twice.

The system was then loaded with the aqueous solution of polysaccharide conjugate and lipids. The right syringe was filled with organic/lipids solution and the left syringe was filled with aqueous polysaccharide conjugate solution. The flow rate needed in order to comply with the chosen ratio was programed into the software and the nanopreciptation process was started:

Flow rates:

| | |
|---|---|
| aqueous phase (mL/min) | 12.8 |
| organic phase (EtOH) (mL/min) | 3.2 |
| TFR | 16 |

Liposomes were harvested as follows: The first 1 ml were discarded (until a good vortex was visualized with no bubble in the microchip). The liposomes were harvested in a conical container and kept at 2-8° C. until buffer exchange step. (Typically no more than 30 min). Post microfluidics system cleaning was the same as the pre-microfluidics process provided above.

| | | | | | | | | Organic Phase | | | | |
|---|---|---|---|---|---|---|---|---|---|---|---|---|
| | | | | | | Aqueous Phase | | | Cationic | | | |
| | | | Final Formulation | | | | Component | | lipid | | PRRP | |
| | | Cationic | (mg/mL) | | | Component | 3 Hib-TT | Total | (final | | Repeat | Cationic |
| | | Lipid | | Hib- | Sample | 2 Maleate | (PRRP, | Lipids | sample | Sol- | Unit | lipid |
| | | (mol %) | Lipids | TT | Name | (mM) pH 6 | μg/mL) | (mg/mL) | (mg/mL) | vents | (mol) | (mol) |
| DDAB | DOPC- | 60% | 10 | 0 | 18INF019_1 | 10 | 0 | 50 | 6.32 | EtOH | 0 | 2.00E−06 |
| N/A | ationic | 0% | 10 | 0.05 | 18INF019_2 | 10 | 62.5 | 50 | 0 | EtOH | 1.45E−07 | N/A |
| DOTAP | Lipids | 60% | 10 | 0.05 | 18INF019_3 | 10 | 62.5 | 50 | 6.55 □ | EtOH | 1.45E−07 | 1.88E−06 |
| DDAB | (%) Chol | 60% | 10 | 0.05 | 18INF019_4 | 10 | 62.5 | 50 | 6.32 | EtOH | 1.45E−07 | 2.00E−06 |
| DDAB | | 60% | 10 | 0 | 18INF019_5 | 10 | 0 | 50 | 6.32 | EtOH | 0 | 2.00E−06 |
| DDAB | | 60% | 10 | 0.05 | 18INF019_6 | 10 | 62.5 | 50 | 6.32 | EtOH | 1.45E−07 | 2.00E−06 |
| DOTAP | | 60% | 10 | 0.05 | 18INF019_7 | 10 | 62.5 | 50 | 6.55 | EtOH | 1.45E−07 | 1.88E−06 |

| | Flow | Flow Aq. | Flow Org | | | Visual | |
|---|---|---|---|---|---|---|---|
| N:P | Total | Phase | phase | | T* | observation* | Sample |
| ratio | mL/min | mL/min | mL/min | Ratio | ° C. | Homogenous | Name |
| NA | 16 | 12.8 | 3.2 | 4 | 27.4 | Turbid +++, homogenous | 18INF019_1 |
| NA | 16 | 12.8 | 3.2 | 4 | 27.7 | Turbid ++, homogenous | 18INF019_2 |
| 13 | 16 | 12.8 | 3,2 | 4 | 27.7 | limpid + | 18INF019_3 |
| 14 | 16 | 12.8 | 3.2 | 4 | 28.3 | Turbid +++, homogenous | 18INF019_4 |
| NA | 16 | 12.8 | 3.2 | 4 | 48.3 | Turbid ++, homogenous | 18INF019_5 |
| 14 | 16 | 22.8 | 3.2 | 4 | 45.8 | Turbid +++, homogenous | 18INF019_6 |
| 13 | 16 | 12.8 | 3.2 | 4 | 48.8 | Limpid without bubble | 18INF019_7 |

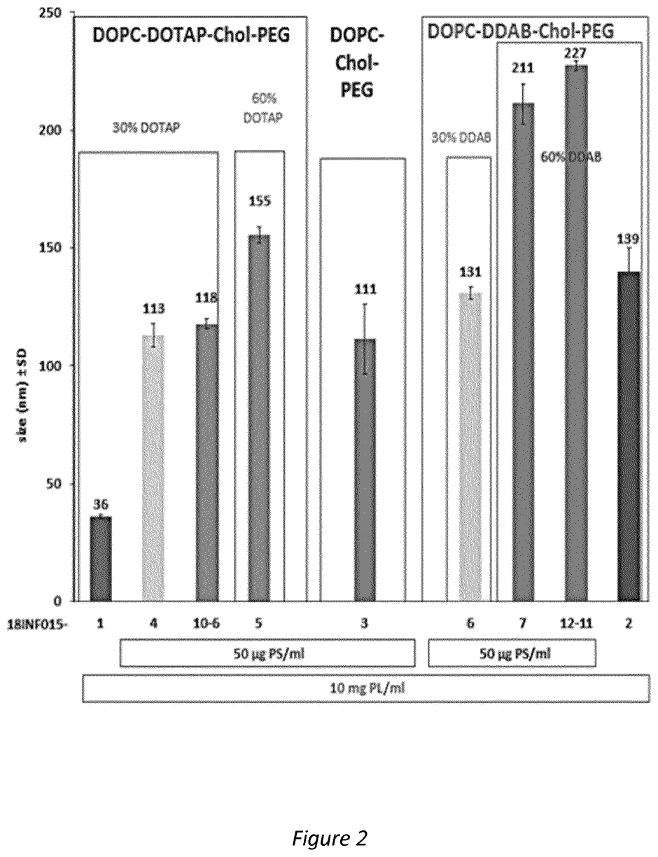

Additional data is provided in FIG. 2 for the following liposomal compositions:

| | Lipid | | | | | |
|---|---|---|---|---|---|---|
| | composition | Final total | | | | |
| | (molar | lipid | Final HibTT | | Flow | Total |
| Sample # | concentration | concentration | concentration | Lipid:Hib | rate ratio | flow rate |
| 18INF015_{—} | %) | (mg/mL) | (μg/mL) | ratio | (aq:org) | (mL/min) |
| 1 | DOPC-DOTAP- | 10 | 0 | NA | 4:1 | 16 |
| | Chol-PEG | | | | | |
| | (34:30:34:2) | | | | | |
| 2 | DOPC-DDAB- | 10 | 0 | NA | 4:1 | 16 |
| | Chol-PEG | | | | | |
| | (4:60:34:2) | | | | | |
| 3 | DOPC-Chol- | 10 | 50 | 200 | 4:1 | 16 |
| | PEG | | | | | |
| | (64:34:2) | | | | | |
| 4 | DOPC-DOTAP- | 10 | 50 | 200 | 4:1 | 16 |
| | Chol-PEG | | | | | |
| | (34:30:34:2) | | | | | |
| 5 | DOPC-DOTAP- | 10 | 50 | 200 | 4:1 | 16 |
| | Chol-PEG | | | | | |
| | (5:60:33:2) | | | | | |
| 6 | DOPC-DDAB- | 10 | 50 | 200 | 4:1 | 16 |
| | Chol-PEG | | | | | |
| | (34:30:34:2) | | | | | |
| 7 | DOPC-DDAB- | 10 | 50 | 200 | 4:1 | 16 |
| | Chol-PEG | | | | | |
| | (4:60:34:2) | | | | | |

### Buffer Exchange

Following μ-fluidics, 10 mL of each sample was dyalised. Dyalisis is performed with Thermoscientific Slide-A-Lyzer™ Dialysis Cassettes, 7K MWCO, 12 mL in 2 L of 10 mM maleate (pH6) under magnetic stirring, the buffer was changed after: 15 min, 30 min, 30 min, 1 h, 1 h and left overnight at 4° C. After the dialysis, the samples were kept at 4° C.

### Liposome Characterisation

Size measurement by DLS (DynaPro PlateReader-II, Wyatt™). The liposome samples were diluted 1:100 in 10 mM maleat buffer. 100 μL of each sample was added in 96-well plate Five measurements are made and the average was calculated.

### Flocculation Assays

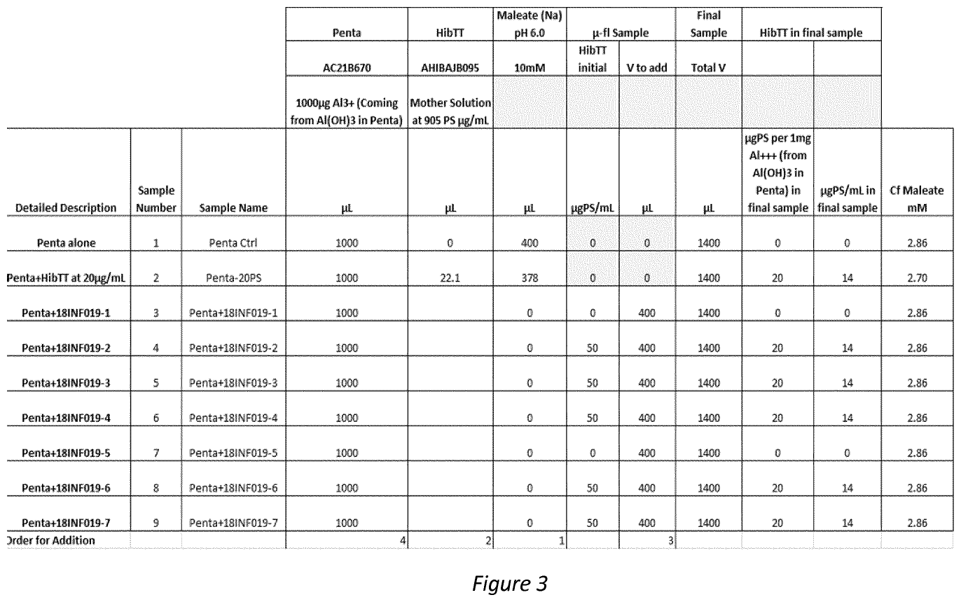

500 μL of Infanrix Penta was mixed with a corresponding volume of liposomes comprising 10 μg of Hib; 10 μg of Hib-TT was added to 500 μL of Infanrix Penta as a reference for comparison. Infanrix Penta is a commercially available vaccine which contains DT, TT, three purified antigens of Bordetella pertussis [pertussis toxoid (PT), pertussis filamentous haemagglutinin (FHA) and pertactin (PRN)] and the purified major surface antigen (HBsAg) of the hepatitis B virus (HBV), adsorbed on aluminium salts. It also contains three types of inactivated polio virus (IPV) (type 1: Mahoney strain; type 2: MEF-1 strain; type 3: Saukett strain). Details are provided in the table in FIG. 3.

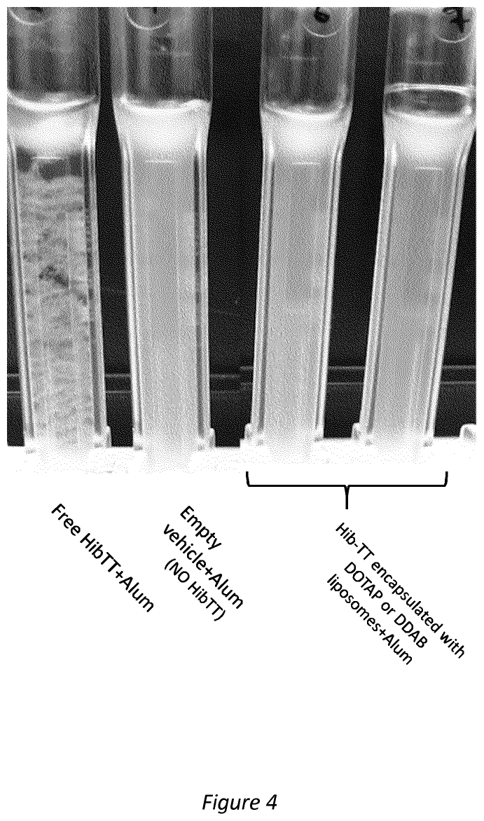

Visual analysis of flocculation was performed as shown in FIG. 4. The images obtained with the Infanrix Penta reference show a smooth suspension with small particles of alum, while those obtained with unencapsulated Hib-TT conjugate show large aggregates of Hib-TT. The incompatibility of the Hib-TT conjugate with Infanrix Penta has been previously observed as significant flocculation when the conjugate is put in contact with the aluminium hydroxide, due to the formation of multiple salt bridges between the Hib polysaccharide and the alum particles.

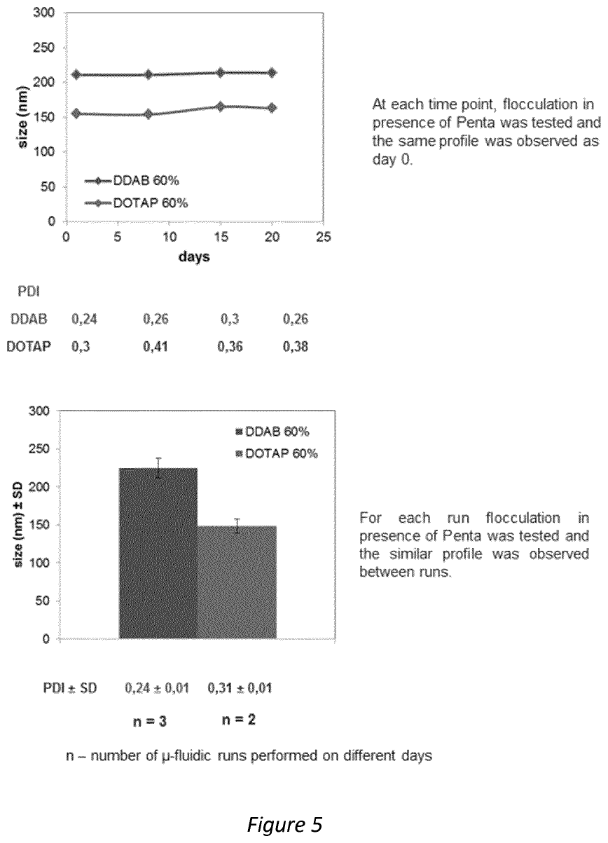

Surprisingly, and in contrast to the results obtained with Hib-TT, HibTT-lipid particles showed complete absence of flocculation when put in contact with pentavalent DTP-based vaccine thus indicating efficient protection of HibTT from interference with alum. Moreover, particle stability was confirmed in terms of preserved hydrodynamic diameter, homogeneity in the absence of alum and absence of flocculation after contact with alum over time (FIG. 5).

### Morphology by Electron Microscopy

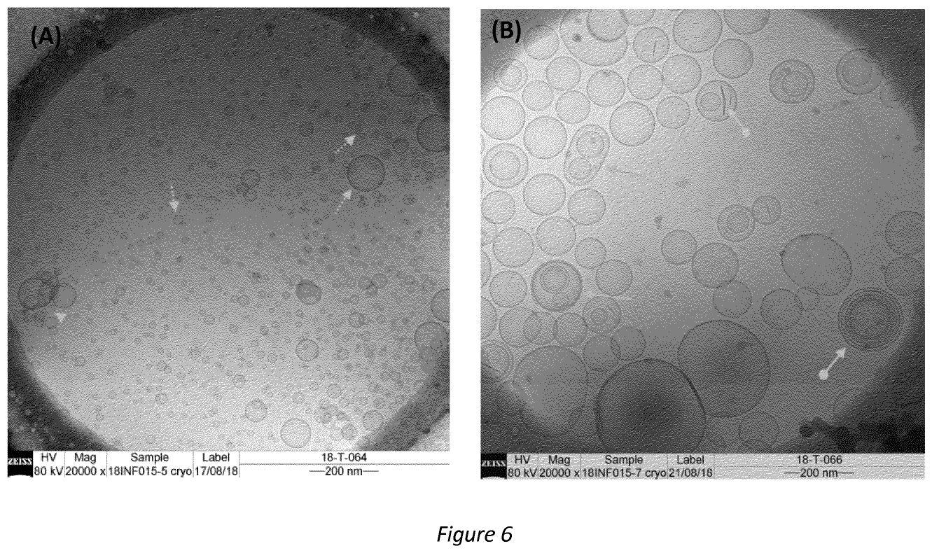

Morphological characterization of lipid nanoparticles containing HibTT by EM negative staining and cryo-EM analysis was carried out. The aim of this experiment is to obtain a morphological assessment of lipid nanoparticles containing polysaccharide conjugates (FIG. 6). The liposomes produced were relatively homogenous in size and shape.

### DDAB Study Design

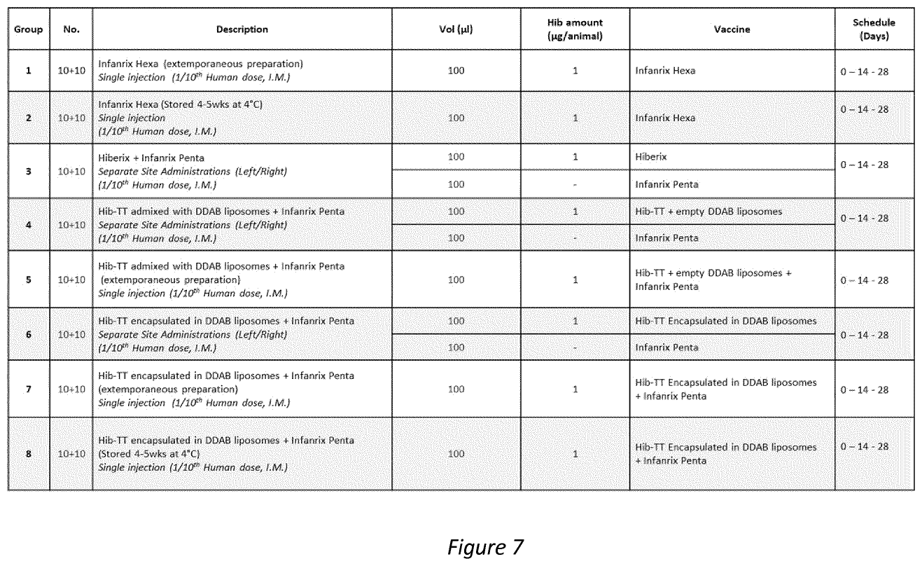

Two studies were performed in parallel, each including eight groups of ten adult rats (FIG. 7). The adult rats (6 week old adult female rat OFA) were immunized by intramuscular route on days 0.14 and 28. Bleeding was performed on day 21 (7PII—Partial bleeding) and day 35 (7PII—final bleeding).

### Immunological Read-Outs

### Humoral Responses—Binding Antibodies by ELISA

Sera from all rats were individually collected seven days after the second and third immunization and tested for the presence of Haemophilus influenzae type b polyribosyl-ribitol-phosphate (PRP) specific IgG antibodies according to the following protocol:

96-well plates were coated with tyraminated PRP (0.5 μg/ml) in a carbonate-bicarbonate buffer (50 mM) and incubated overnight at 4° C. Rat sera were diluted at 1/10 (7PII) and 1/40 (7PII) in PBS-Tween 0.05% and serially diluted in the wells from the plates (12 dilutions, step ½). An anti-Rat IgG (H+L) polyclonal antibody coupled to the peroxidase was added (1/750 dilution). Colorimetric reaction was observed after the addition of the peroxidase substrate (OPDA), and stopped with HCI (1 M) before reading by spectrophotometry (wavelengths: 490-620 nm). For each serum tested and standard added on each plate, a 4-parameter logistic curve was fitted to the relationship between the OD and the dilution (Softmaxpro). This allowed the derivation of each sample titer expressed in STD titers.

Humoral Responses—Serum Bactericidal Activity (SBA) Against Haemophilus influenzae Type b

The Hib 20.750 strain was subcultured onto “Chocolate Haemophilus 2” (Bioérieux) agar plates and incubated overnight at 36° C. without CO₂. After approximately 15 h of culture on chocolate plate, a liquid culture was prepared. Bacteria were scraped from plates and suspended in 12.5 ml of enriched TSB (Tryptic Soy Broth +1% Polyvitex +1% horse serum +0.1% NAD +0.1% hemin) in a Wiame flask until an OD of 0.05 at 470 nm was obtained. After approximately 3 h of incubation at 37° C. with shaking at 200 rpm, when the OD was around 0.4 at 470 nm, the bacteria were diluted in bactericidal buffer (HBSS-BSA 1%) in order to yield a bacterial preparation of 1.10⁴ CFU/ml. The bacterial preparation was then mixed at equal volume with either active or heat-inactivated precolostral calf serum complement 15%. Twenty-five μl/well of the mixes together with 25 μl/well of serial 2-fold dilutions in HBSS-BSA 1% of heat-inactivated pooled sera or of pooled sera from all Hib group (positive control) or of pooled sera from the LAS (negative control) were added to sterile flat-bottom microtiter plates. Microplates were incubated at 37° C. with shaking at 210 rpm for 45 minutes. After incubation, 10 μl of each well were plated on “Chocolate Haemophilus 2” (Bioérieux) agar plates using the tilt method (allowing the mixture to flow 8 to 10 cm in lanes down the plate) to determine the number of CFU. Colonies were counted after overnight incubation at 33° C. without CO₂. SBA titer was determined as the reciprocal of the highest dilution resulting in 50% killing compared to the average colony count for active complement-only control wells.

### Results

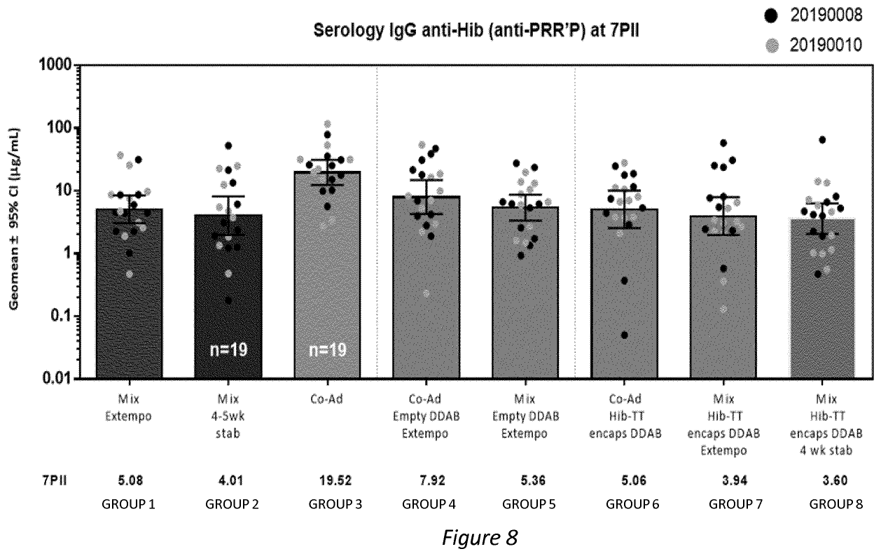

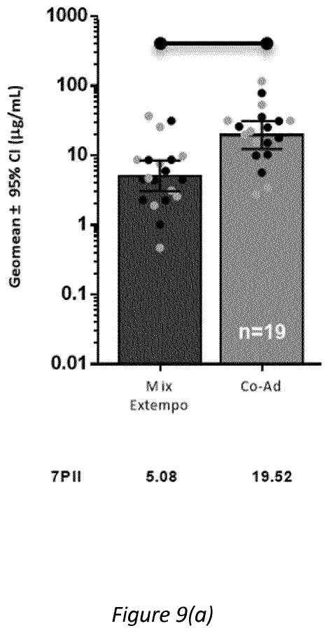

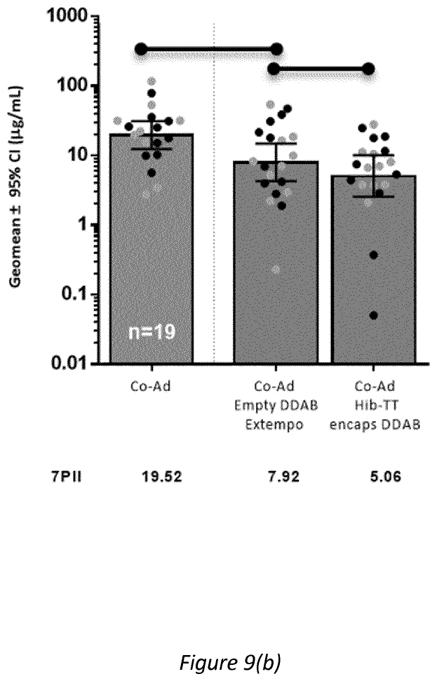

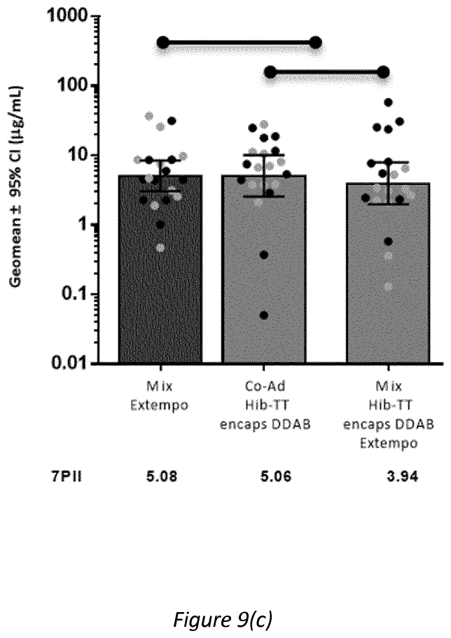

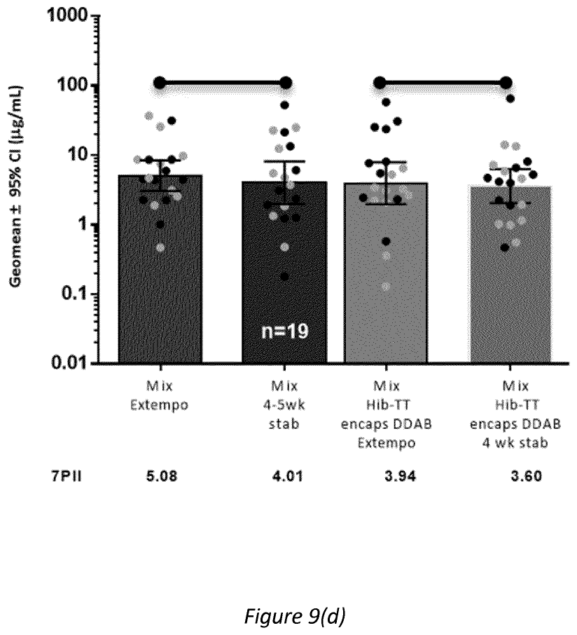

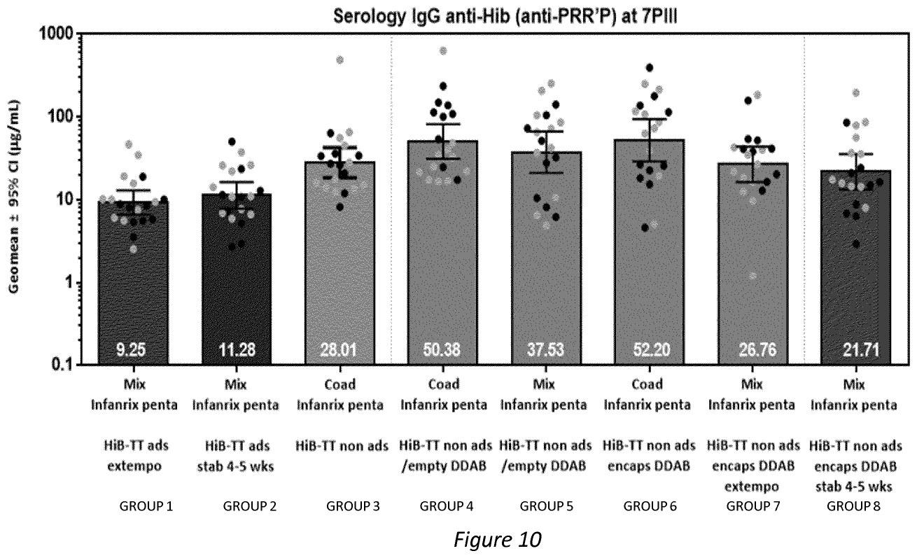

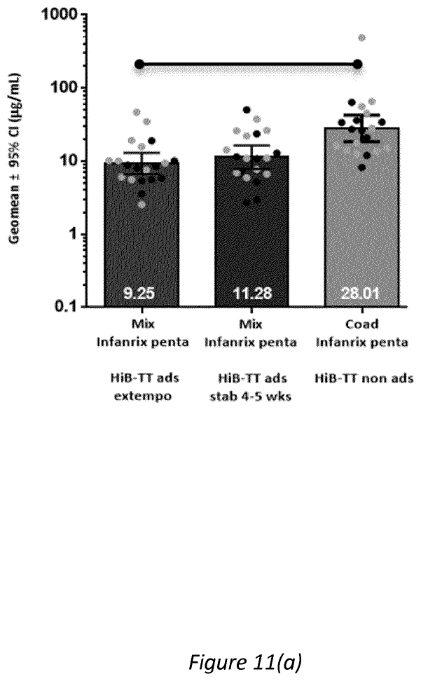

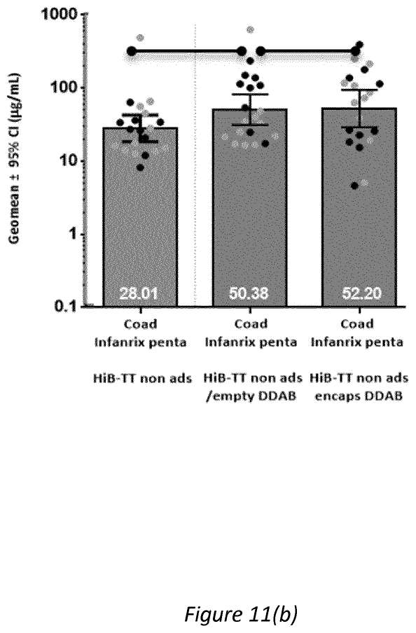

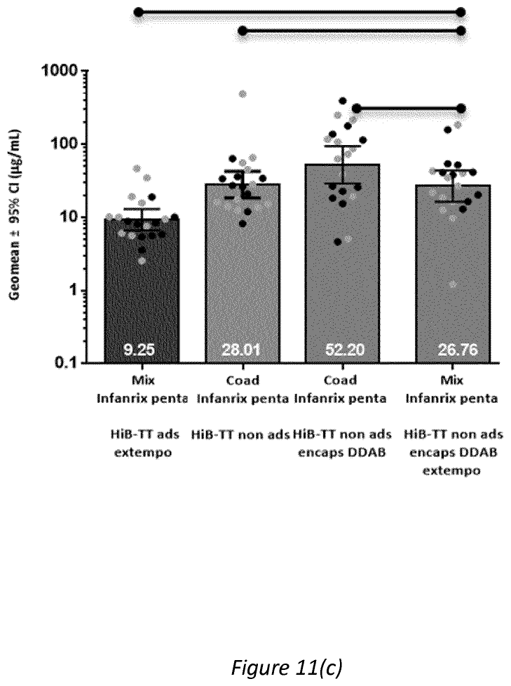

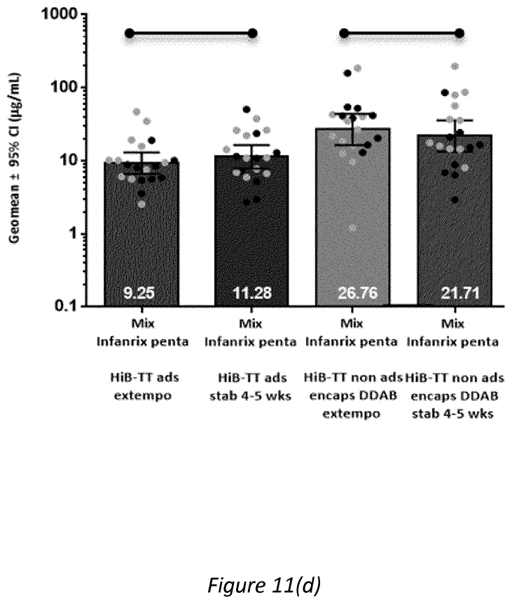

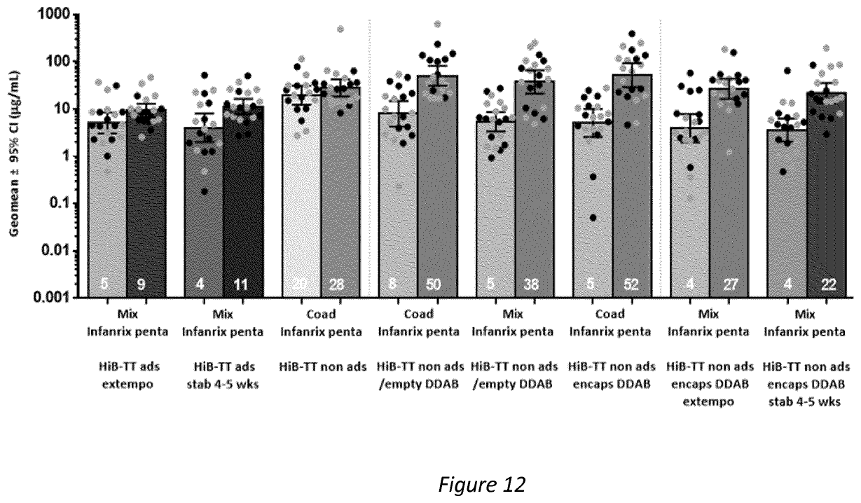

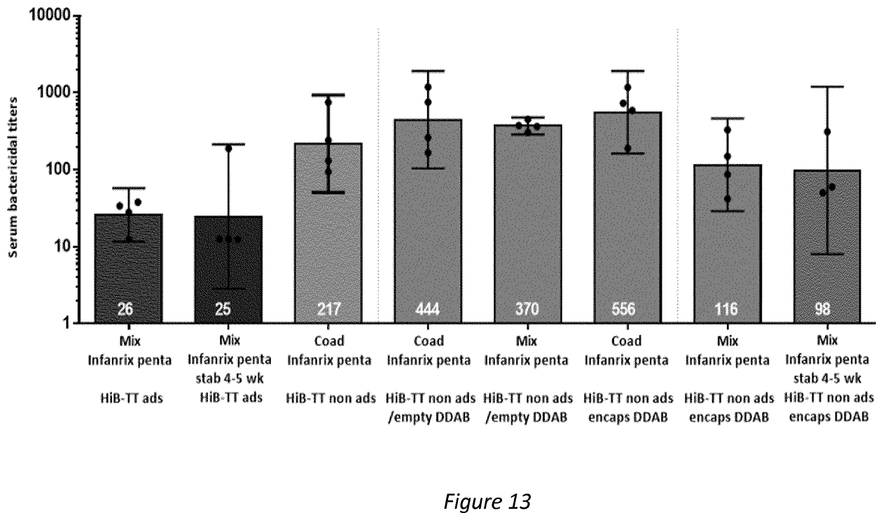

The aim of this experiment was to evaluate whether any interference that might be observed with Hib-TT when mixed with a pentavalent DTaP-IPV-HepB vaccine (Infanrix Penta) is seen when Hib-TT is encapsulated in DDAB-based liposomes. The results of the experiments are provided in FIGS. 8 to 13.

At 7PII, groups immunised with DDAB-based liposomes exhibited reduced immune responses compared to the control group suggesting interaction between DDAB and Hib-TT. However, at 7PIII no reduction in immune response was observed. A complementary in vitro evaluation did not show any TLR4 signaling activation by DDAB alone nor by Hib-TT encapsulated in DDAB-based liposomes (data not shown).

These results indicate that, the use of DDAB-based liposomes for encapsulation may be an effective strategy to prevent interaction of encapsulated components with non-encapsulated components in a composition, for example to ‘protect’ Hib conjugates from cleavage or degradation in the presence of aluminium hydroxide.

### DOTAP Study Design

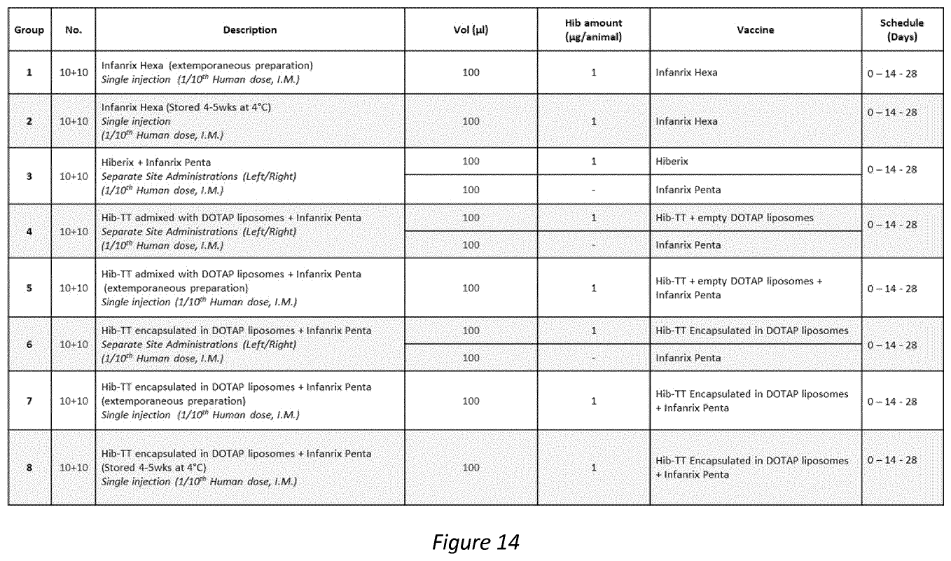

Two studies were performed in parallel, each including eight groups of ten adult rats (FIG. 14). The adult rats (6 week old adult female rat OFA) were immunized by intramuscular route on days 0,14 and 28. Bleeding was performed on day 21 (7PII—Partial bleeding) and day 35 (7PIII—final bleeding). Immunological read-outs were performed as before.

### Results

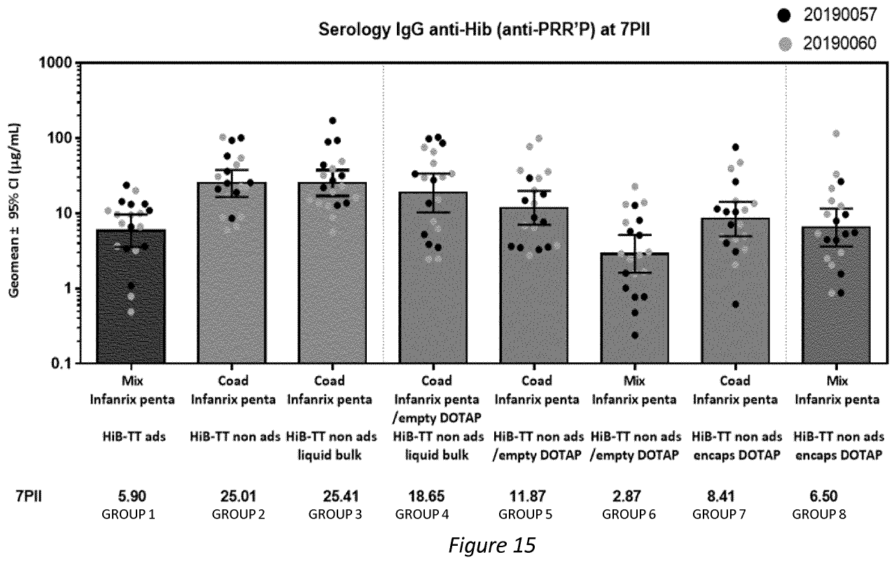

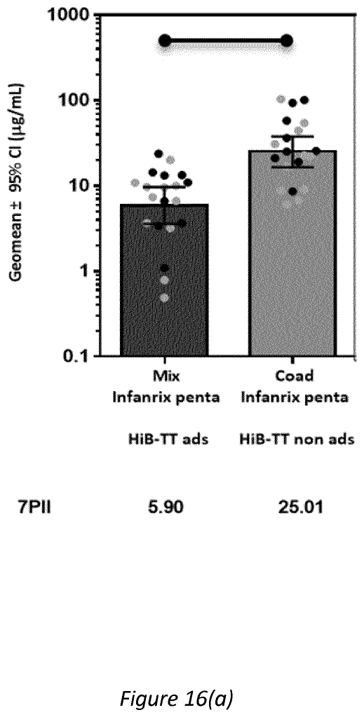

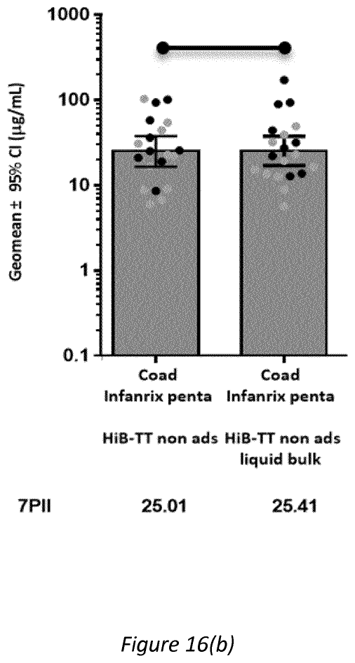

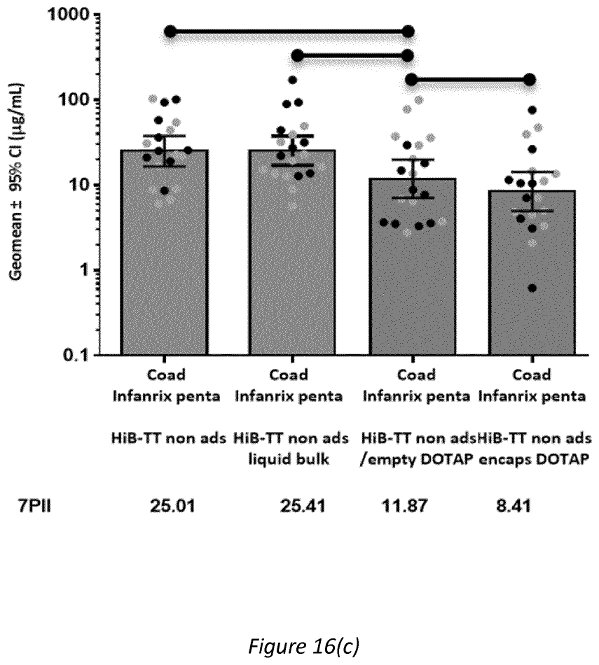

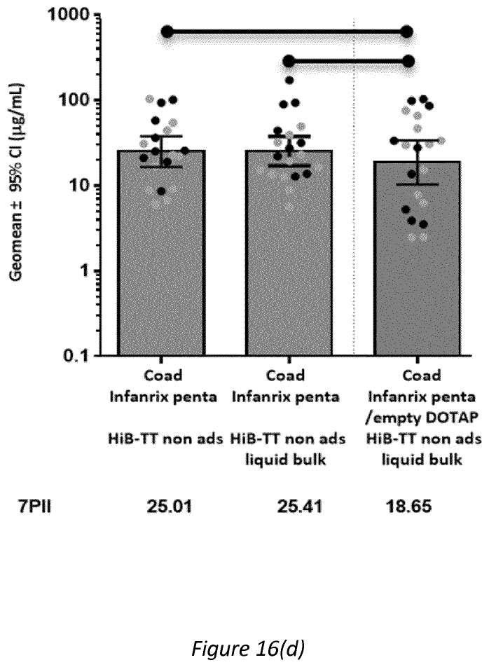

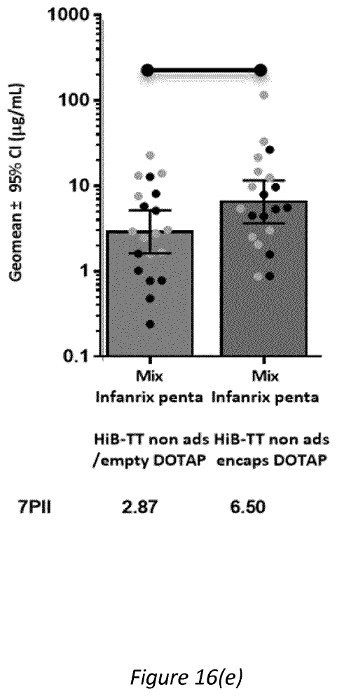

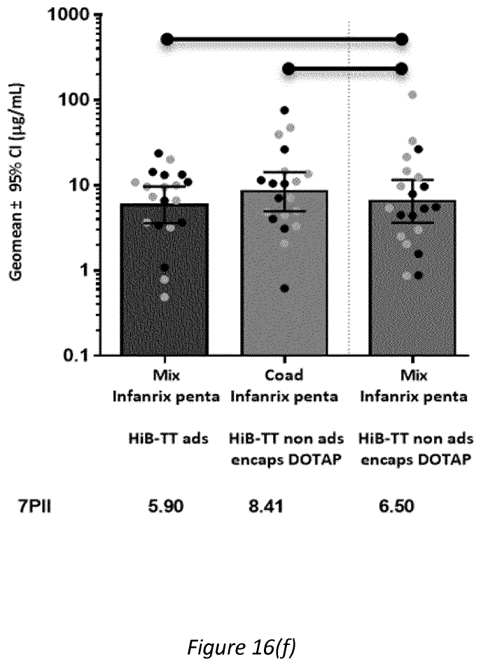

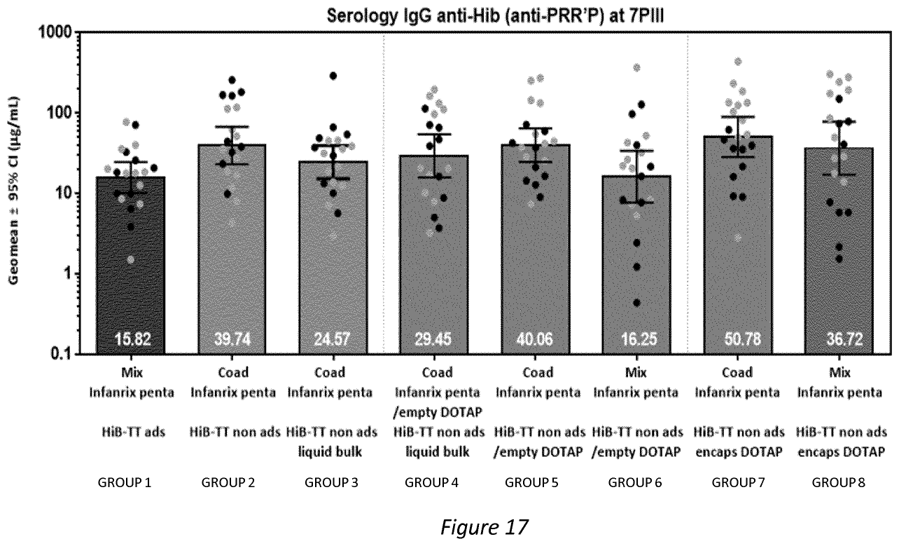

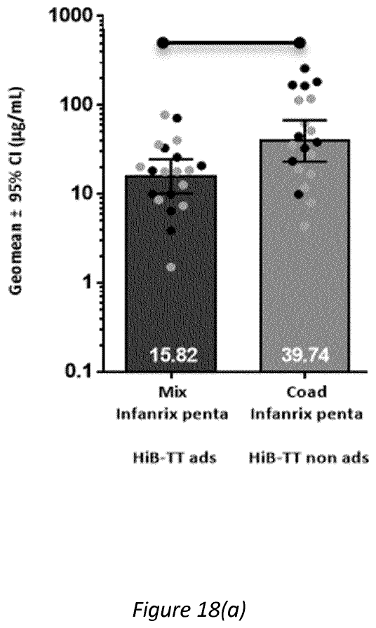

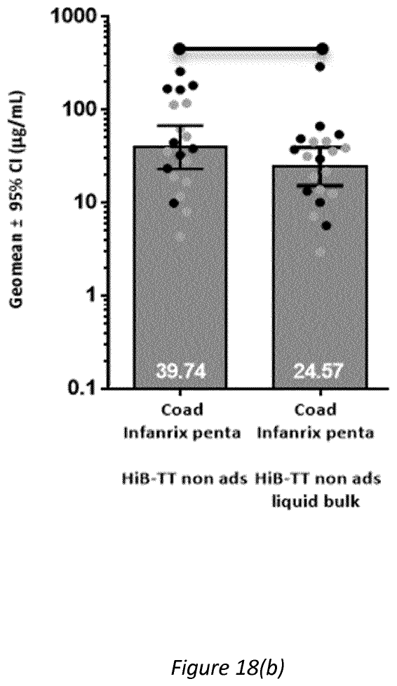

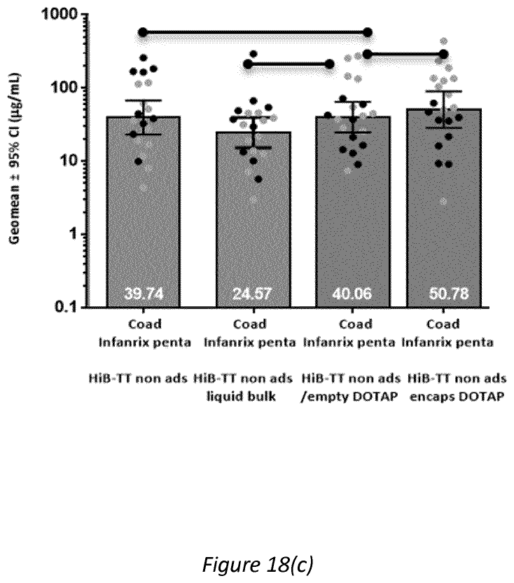

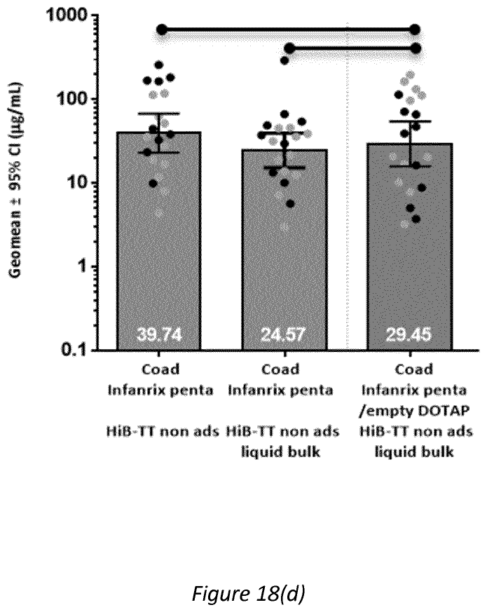

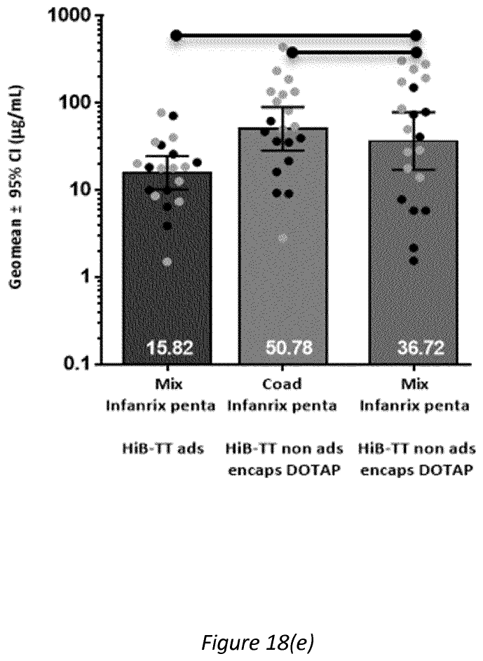

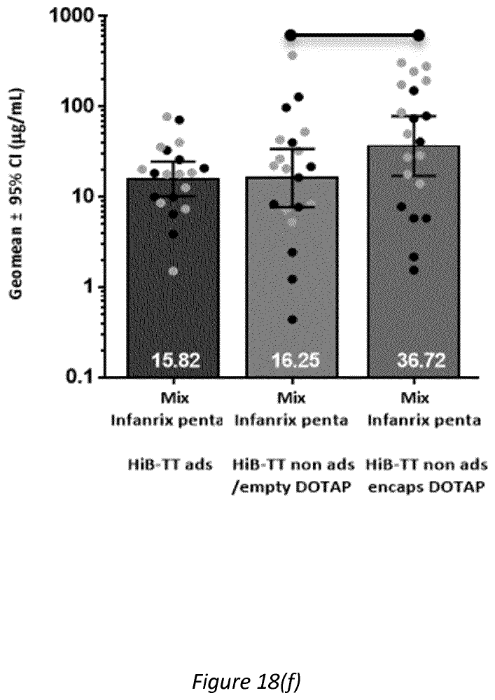

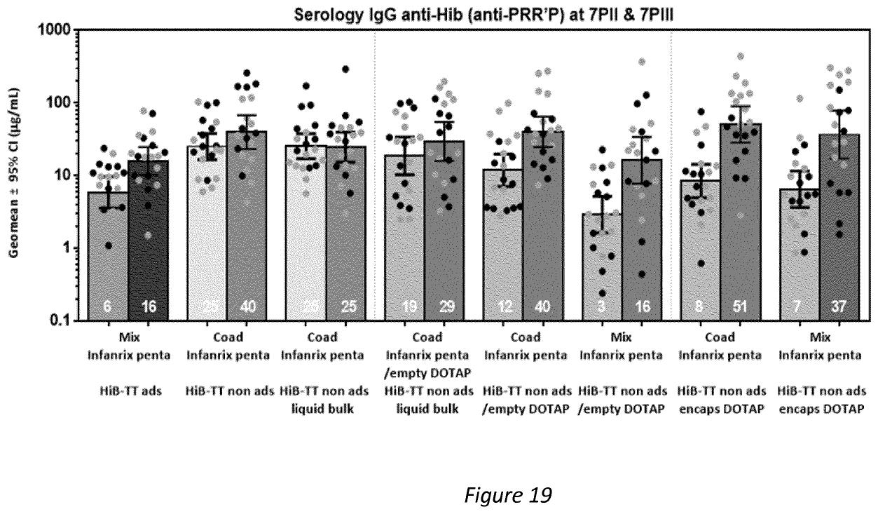

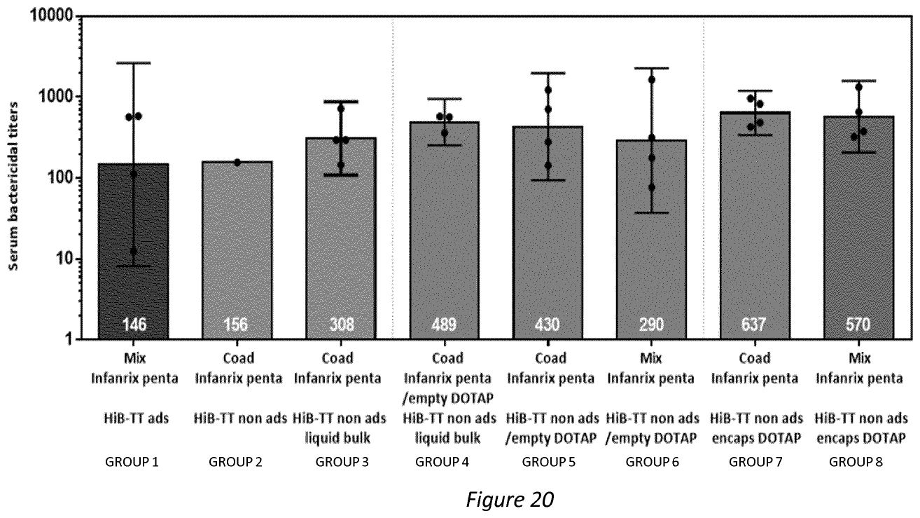

The aim of this experiment was to evaluate whether any interference that might be observed with Hib-TT when mixed with a pentavalent DTaP-IPV-HepB vaccine (Infanrix Penta) is seen when Hib-TT is encapsulated in DOTAP-based liposomes. The results of the experiments are provided in FIGS. 15 to 20.

As before, at 7PII, groups immunised with DOTAP-based liposomes exhibited reduced immune responses compared to the control group suggesting interaction between DOTAP and Hib-TT. However, at 7PIII no reduction in immune response was observed

These results indicate that, the use of DOTAP-based liposomes for encapsulation may be an effective strategy to prevent interaction of encapsulated components with non-encapsulated components in a composition, for example to ‘protect’ Hib conjugates from cleavage or degradation in the presence of aluminium hydroxide.

1. A nanoparticle, for example a liposome, comprising a lipid component and a polysaccharide conjugate component, said lipid component comprising (i) DOPC, (ii) a cationic lipid, (iii) Cholesterol and (iv) DSPE-PEG.

2. The nanoparticle according to claim 1 wherein the cationic lipid is selected from the group consisting of DOTAP and DDAB.

3. The nanoparticle of claim 2, wherein the lipid component comprises DOPC:DOTAP:Cholesterol:DSPE-PEG in a ratio (molar concentration %) of about 4:60:34:2.

4. The nanoparticle of claim 2, wherein the lipid component comprises DOPC:DDDAB:Cholesterol:DSPE-PEG in a ratio (molar concentration %) of about 4:60:34:2.

5. The nanoparticle of claim 1 wherein the polysaccharide conjugate component comprises polyribosylribitol phosphate.

6. The nanoparticle of claim 5, wherein the polyribosylribitol phosphate is derived from Haemophilus influenzae serotype B.

7. The nanoparticle of claim 5, wherein the polyribosylribitol phosphate is a synthetic polysaccharide capable of cross-reacting with antibodies specific to polyribosylribitol phosphate derived from Haemophilus influenzae serotype B.

8. The nanoparticle of claim 1 wherein the polysaccharide conjugate component is derived from Neisseria meningitidis, particularly serotype A.

9. The nanoparticle of claim 5, wherein the polysaccharide conjugate component comprises either (i) a carrier protein selected from the group consisting of diphtheria toxoid (DT), CRM197, tetanus toxoid (TT) and OMPC or (ii) an outer membrane vesicle carrier.

10. A plurality of nanoparticles according to claim 1, wherein the average nanoparticle size is from about 150 nm to about 250 nm.

11. A plurality of nanoparticles according to claim 1, having a polydispersity index in the range of from 0.05 to 0.45.

12. An aqueous composition comprising the plurality of nanoparticles according to claim 10.

13. The aqueous composition of claim 12, wherein the polysaccharide conjugate is present at a concentration of about 50 μg/ml or less.

14. The aqueous composition of claim 12, wherein the lipid component is present at a concentration of about 10 mg/ml.

15. The aqueous composition of claim 12, wherein the lipid:polysaccharide ratio (w/w) is about 200:1.

16. The aqueous composition of claim 12 which is an immunogenic composition.

17. The aqueous composition of claim 16 further comprising an antigen selected from the group consisting of diphtheria toxoid, tetanus toxoid, inactivated polio virus (IPV), hepatitis B surface antigen, pertussis toxoid (PT), pertactin, FHA and fimbrial protein.

18. The nanoparticle of claim 1, for use in a method of raising an immune response in a mammal.

19. A method of manufacturing a nanoparticle according to claim 1, using a microfluidic device, the method comprising the steps of mixing in the device a first solution comprising an aqueous solvent and a polysaccharide conjugate and a second solution comprising an organic solvent and (i) DOPC, (ii) the cationic lipid, for example, DOTAP or DDAB, (iii) Cholesterol and (iv) DSPE-PEG.
